SEQUENCE LISTING (7 sequences)

SEQ ID NO: 1 - protein, 349 aa, Artificial Sequence
  PUF531
GRSRLLEDFRNNRYPNLQLREIAGHIMEFSQDQHGSRFIELKLERATPAERQLVFNEILQAAYQLMVDVFGNYVIQKFFEFGSLEQKLALAERIRGHVLSLALQMYGSRVIEKALEFIPSDQQNEMVRELDGHVLKCVKDQNGNHVVQKCIECVQPQSLQFIIDAFKGQVFALSTHPYGSRVIERILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLG

SEQ ID NO: 2 - protein, 349 aa, Artificial Sequence
  PUF mRNA loc/PUFeng
GRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGSYVIEHVLEHGRPEDKSKIVAEIRGNVLVLSQHKFANNVVQKCVTHASRTERAVLIDEVCTMNDGPHSALYTMMKDQYANYVVQKMIDVAEPGQRKIVMHKIRPHIATLRKYTYGKHILAKLEKYYMKNGVDLG

SEQ ID NO: 3 - protein, 644 aa, Artificial Sequence
  PUFx2
GRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDSHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLG

SEQ ID NO: 4 - protein, 186 aa, Artificial Sequence
  Cas6
DHYLDIRLRPDPEFPPAQLMSVLFGKLHQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRAQSTGQHFRLFIRHGPLQVTAEEGGFTCYGLSKGGFVPWF

SEQ ID NO: 5 - protein, 37 aa, Artificial Sequence
  His aptamer
GGGTATATTGGCGCCTTCGTGGAATGTCAGTGCCTGG

SEQ ID NO: 6 - protein, 36 aa, Artificial Sequence
  TAT aptamer
TGCTTCGAACTAGGGCAAACGGCCAGCTAGCGAAGC

SEQ ID NO: 7 - protein, 464 aa, Homo sapiens
MASESGKLWGGRFVGAVDPIMEKFNASIAYDRHLWEVDVQGSKAYSRGLEKAGLLTKAEMDQILHGLDKVAEEWAQGTFKLNSNDEDIHTANERRLKELIGATAGKLHTGRSRNDQVVTDLRLWMRQTCSTLSGLLWELIRTMVDRAEAERDVLFPGYTHLQRAQPIRWSHWILSHAVALTRDSERLLEVRKRINVLPLGSGAIAGNPLGVDRELLRAELNFGAITLNSMDATSERDFVAEFLFWASLCMTHLSRMAEDLILYCTKEFSFVQLSDAYSTGSSLMPQKKNPDSLELIRSKAGRVFGRCAGLLMTLKGLPSTYNKDLQEDKEAVFEVSDTMSAVLQVATGVISTLQIHQENMGQALSPDMLATDLAYYLVRKGMPFRQAHEASGKAVFMAETKGVALNQLSLQELQTISPLFSGDVICVWDYGHSVEQYGALGGTARSSVDWQIRQVRALLQAQQA

(19) United States
(12) Patent Application Publication
(10) Pub. No.: US 2021/0386868 A1
(43) Pub. Date: Dec. 16, 2021

(54) EXTRACELLULAR VESICLES FOR REPLACEMENT OF UREA CYCLE PROTEINS & NUCLEIC ACIDS

(71) Applicant: Evox Therapeutics Ltd, Oxford (GB)

(72) Inventors: Justin HEAN, Oxford (GB); Harsh AMIN, Abingdon (GB); Virginia CASTILLA LLORENTE, Oxford (GB)

(21) Appl. No.: 17/281,535

(22) PCT Filed: Nov. 18, 2019
(86) PCT No.: PCT/EP2019/081672

§ 371 Date: Mar. 30, 2021

(30) Foreign Application Priority Data
Nov. 16, 2018 (GB) 1818761.7
Jan. 7, 2019 (GB) 1900197.3

(51) Int. Cl. A61K 47/69; C07K 14/705

(52) U.S. Cl. CPC A61K 47/6901; C07K 14/705

(57) ABSTRACT

The present invention relates to engineered extracellular vesicles (EVs) as a novel therapeutic approach to treating urea cycle disorders. More specifically, the invention relates to the use of various protein engineering and nucleic acid engineering strategies for improving loading of urea cycle proteins or nucleic acids encoding urea cycle proteins into EVs and targeting of the resultant EVs to tissues and organs of interest.

### TECHNICAL FIELD

The present invention relates to engineered extracellular vesicles (EVs) as a novel therapeutic approach to the treatment of urea cycle disorders. More specifically, the invention relates to the use of various protein and nucleic acid engineering strategies for improving loading of urea cycle-related proteins and/or nucleic acids and targeting of the resultant EVs to tissues and organs of interest.

### BACKGROUND

Genetic defects in the enzymes involved in the urea cycle leads to faulty metabolism of the nitrogen-containing compound urea. Mutations lead to deficiencies of the various enzymes and transporters involved in the urea cycle and cause urea cycle disorders. If individuals with a defect in any of the urea cycle enzymes or transporters ingest amino acids beyond what is necessary for the minimum daily requirements the ammonia that is produced will not be able to be converted to urea. These individuals can experience hyperammonemia or the build-up of a toxic cycle intermediate.

Urea cycle disorders (UCD) are genetic errors of metabolism caused by a deficiency in enzymes or mitochondrial transport proteins involved in the production of urea, resulting in accumulation of toxic levels of ammonia in the blood (hyperammonemia). The most common urea cycle disorders are:
N-Acetylglutamate synthase deficiency
Carbamoyl phosphate synthetase deficiency
Ornithine transcarbamoylase deficiency
Citrullinemia (deficiency of argininosuccinic acid synthase)
Argininosuccinic aciduria (deficiency of argininosuccinic acid lyase)
Argininemia (deficiency of arginase)
Hyperornithinemia, hyperammonemia, homocitrullinuria (HHH) syndrome (deficiency of the mitochondrial ornithine transporter)
Citrullinemia II (deficiency of citrin, an aspartate glutamate transporter)
Lysinuric protein intolerance (mutation in y+L amino acid transporter 1)
Orotic aciduria (deficiency in the enzyme uridine monophosphate synthase UMPS)

UCD subtypes include those caused by an X-linked mutation and corresponding deficiency in ornithine transcarbamylase (OTC) and those caused by autosomal recessive mutations with corresponding deficiencies in argininosuccinate synthetase (ASS), carbamyl phosphate synthetase (CPS), argininosuccinate lyase (ASL), arginase (ARG), N-acetylglutamate synthetase (NAGS), ornithine translocase (HHH), and aspartate glutamate transporter (CITRIN). These are rare diseases, with an overall estimated incidence in the United States of approximately 1 in every 35,000 live births. UCD is suspected when a subject experiences a hyperammonemic event with an ammonia level >100 μmol/L accompanied by signs and symptoms compatible with hyperammonemia in the absence of other obvious causes and generally confirmed by genetic testing.

ASA as well as the other UCDs are considered as rare genetic disorders. ASA is characterised by deficiency or lack of the enzyme argininosuccinate lyase (ASL). ASL is central to two metabolic pathways: i) the liver-based urea cycle, which detoxifies ammonia, and ii) the citrulline-nitric oxide cycle, which synthesises nitric oxide from L-arginine. Patients with ASL deficiency can present either shortly after birth or later in life and are characterised by hyperammonaemia and a multi-organ disease with a severe neurological phenotype. There is currently an unmet medical need for these patients as ASA is the second most common urea cycle disorder, with merely symptomatic treatment available today.

The severity and timing of UCD presentation vary according to the severity of the deficiency, which may range from minor to extreme depending on the specific enzyme or transporter deficiency, and the specific mutation in the relevant gene. UCD patients may present in the early neonatal period with a catastrophic illness, or at any point in childhood, or even adulthood, after a precipitating event such as infection, trauma, surgery, pregnancy/delivery, or change in diet. Acute hyperammonemic episodes at any age carry the risk of encephalopathy and resulting neurologic damage, sometimes fatal, but even chronic, sub-critical hyperammonemia can result in impaired cognition. UCDs are therefore associated with a significant incidence of neurological abnormalities and intellectual and developmental disabilities over all ages. UCD patients with neonatal-onset disease are especially likely to suffer cognitive impairment and death compared with patients who present later in life.

There are currently no cures for urea cycle disorders, as such, this represents a significant unmet medical need. The treatment of disorders related to the urea cycle is a lifelong process aimed at managing symptoms. Some patients are considered for liver transplantation but the main disease management strategy is through dietary restrictions to reduce dietary protein intake. Pharmacological treatment with ammonia scavenging compounds is widely used to treat UCDs but is not curative and merely manages the symptoms. Sodium phenylbutyrate or buphenyl, sodium benzoate, oral lactulose and neosporin can help scavenge ammonia or prevent ammonia production by bacteria in the colon, these treatments are however often associated with severe side effects and have a relatively small therapeutic window. Multivitamins, calcium and antioxidant supplements are also prescribed in many cases.

Pharmaceutical grade L-citrulline supplements are used for carbamoylphosphate synthetase (CPS) and ornithine transcarbamylase (OTC) deficiency and L-arginine is used in case of argininosuccinate synthase deficiency and citrullinemia to catalyze the enzymes in urea cycle and support optimum ammonia removal. Antacids are often used to relieve gastrointestinal side effects of these drugs, such as acid reflux and stomach ache.

Biopharmaceuticals such as protein biologics (of which one important but often insufficient class of drugs for UCDs are enzyme replacement therapies (ERTs)) and RNA therapeutics may provide a more efficacious alternative means to treat urea cycle disorders. ERTs, however, although active in the treatment of enzyme deficiencies involving the lysosomal compartments are not a viable option for UCDs, as the ERTs cannot gain access to the intracellular environment. mRNA suffers from some of the same issues, namely that access to the intracellular space is severely restricted by the plasma membrane.

Exosomes have been shown to be excellent carriers of various types of biomolecular cargo and are believed to cross the blood brain barrier, however, their actual utility for in vivo delivery of therapeutic proteins and/or mRNA is not trivial and requires thoughtful vesicular engineering.

Nucleic acid-based therapeutics are approaching clinical utility at a rapid pace. Gene therapies, mRNA-based therapies, short oligonucleotide- and siRNA-based therapeutics are just some examples within the plethora of modalities within the RNA therapeutics landscape. As naked nucleic acids, typically RNA, are difficult to deliver in vivo due to rapid clearance, nuclease activity, lack of organ-specific distribution, and low efficacy of cellular uptake, specialized delivery vehicles are usually obligatory as a means of achieving bioactive delivery. This is both the case for hepatic and non-hepatic targets and for high-molecular weight RNA therapeutics such as mRNA and gene therapy.

The EV loading technologies of the prior art are typically very inefficient at loading either protein or nucleic acids (NAs) into the EVs. Firstly, loading systems of the prior art result in variable loading of EVs with either protein and/or NA cargo, and, secondly, those EVs that are loaded are loaded with small numbers of protein and/or NA copies per vesicle (the disadvantages associated with these issues are discussed in more detail below).

Prior art loading technologies normally rely on exogenous loading of protein therapeutics cargo or NA agents into EVs. Conventionally, the protein is produced separately from the EVs and loaded, usually by electroporation or transfection, into separately produced and purified EVs. This method has a number of disadvantages: (i) the cost of goods for producing EVs and therapeutic cargo separately can be prohibitive for commercialization, (ii) exogenous loading techniques can have a negative impact on the integrity and function of the EVs per se, (iii) purification and downstream processing can be difficult and labour-intensive with multiple steps, and (iv) complex proteins loaded exogenously can exhibit problems with conformational changes and unstable and/or incorrect post-translational modifications, potentially leading to reduced activity.

Similarly, NA loading systems of the prior art do typically not achieve functional, bioactive, delivery of the nucleic acid cargo probably because of (i) the loading of mRNAs and other coding RNA molecules is inefficient and variable, (ii) the mRNAs delivered by the prior art technologies are not translated once they reach cells because the nucleic acid is not released from the EVs, and (iii) occasionally an suboptimal EV-producing cell source is used. One such prior art technology is the so called TAMEL system described in in U.S. Ser. No. 14/502,494. The TAMEL system suffers from all of the abovementioned disadvantages and is furthermore suffering from the fact that the system relies on a bacteriophage-derived RNA-binding protein which can cause unwanted immune reactivity.

The TAMEL system is thus not suitable for loading into EVs and subsequent delivery of clinically relevant quantities of bioactive nucleic acids, primarily because of lack of efficient loading and delivery and in part because of immunotoxicity, which would be especially problematic in the context of liver diseases due to the partly hepatosplenic biodistribution pattern of EVs.

These variable and low levels of loading combined with the fact that what little nucleic acid that is loaded is then unlikely to be released and therefore not bioactive or that what little protein is actually loaded is not suitably post translationally modified or not properly folded into the optimal conformation for activity means that the prior art systems have many disadvantages and are not suitable for loading and delivery of clinically relevant quantities of bioactive nucleic acids or bioactive proteins. The present invention overcomes these significant disadvantages and allows for bioactive therapeutic delivery in a non-toxic fashion to the liver and other tissues and organs affected by UCDs.

### SUMMARY OF INVENTION

The present invention relates to engineered extracellular vesicles (EVs) as a novel therapeutic approach to treating urea cycle disorders. More specifically, the invention relates to the use of various protein engineering and nucleic acid engineering strategies for improving loading of urea cycle proteins or nucleic acids encoding urea cycle proteins into EVs and targeting of the resultant EVs to tissues and organs of interest in a non-toxic fashion, in particular to the liver and other tissues and organ systems affected by UCDs.

It is hence an object of the present invention to overcome the above-identified problems associated with engineering of EVs, and to apply these EVs in a completely novel field, namely for the treatment of UCDs. The present invention addresses several of the key aspects of EV-based therapeutics for UCDs, namely packing and loading of complex, protein drug cargos and/or nucleic acid drug cargos into the EVs; optimization of the pharmacokinetics of the EVs themselves; harnessing of the regenerative effects of the EVs; and bioactive delivery of the drug cargo (in this case urea cycle proteins and/or nucleic acids encoding urea cycle proteins) into target cells in vivo.

The present invention achieves this by utilizing novel EV engineering technology to package and load, in a bioactive state and configuration, the complex and often very large urea cycle proteins or NAs (such as mRNA) encoding urea cycle proteins needed to treat UCDs.

Furthermore, the present invention overcomes the problems associated with NA cargo loading and release by utilizing novel EV engineering technology to load and release NA cargo in the appropriate tissues or organs. This is achieved by advanced engineering of polypeptide and polynucleotide constructs to ensure not only highly efficient loading into EVs but also effective release of the NA in question. This is achieved by providing an extracellular vesicle (EV) comprising at least one fusion polypeptide comprising at least one nucleic acid (NA)-binding domain and at least one EV enrichment polypeptide. The NA-binding domain may advantageously be present in several copies, and each NA cargo molecule may also be present in multiple copies with each and every copy having a plurality of binding sites for the NA-binding domain. Importantly, the NA-binding domains which form part of the fusion polypeptide and which are responsible for the interaction with the NA cargo molecule are releasable NA-binding domains, meaning that their binding of the NA cargo molecule is a reversible, releasable interaction. The releasable nature of the binding between the NA-binding domain and the NA cargo molecule is particularly advantageous as the present inventors have realized that overexpression of the NA cargo molecule in EV-producing cells allows for sufficiently high local concentrations to enable interaction between the NA-binding domain and the NA cargo molecule, while the lower concentration of the NA binding molecule in the target location (such as inside a target cell) allows for efficient release of the NA molecule, enabling its bioactive delivery.

Furthermore, the present invention also involves the serendipitous selection and profiling of EVs with particular molecular characteristics from cell sources that provide an optimal balance between suitable pharmacokinetics and regenerative properties, as well as providing EVs comprising additional protein and nucleic acid components with therapeutic activity in various UCDs. The inventors of the present invention have realized that EVs represent an optimal delivery vehicle for UCD enzymes and/or transporters, partly due to their biodistribution patterns and partly as a result of some of their native components, such as heat shock proteins, which help maintain cargo proteins in their optimal bioactive conformation, and other regenerative components. Various cell sources are also proving to be preferable for producing UCD protein/nucleic acid loaded EVs, and therefore, the present invention provides for a novel approach to these typically untreatable diseases. Importantly, the present invention approaches the problem of tackling UCDs from a completely novel angle. UCDs are typically only address with small molecule approaches to scavenge or reduce the toxic accumulation of substrate. In recent years, tentative attempts have been made to try to address this group of diseases using gene therapy. The unexpected realization that EVs and exosomes can constitute an efficient delivery modality is an important aspect of the present invention, enabled by innovative loading and delivery technology for protein or NA replacement therapeutic cargo. EVs may also constitute a highly suitable complementary therapeutic intervention alongside or after virus-mediated gene therapy. The good safety and tolerability profile of EVs enable chronic repeat treatment, which is of considerable importance in gene therapy settings where target organ turnover results in loss of the virally delivered transgene over time, requiring topping up of the NA or protein in question which can be achieved using EV-mediated UCD therapies.

In a first aspect, the present invention thus relates to an extracellular vesicle (EV) for replacement of urea-cycle proteins. This is achieved by using engineered EVs loaded with at least one urea-cycle protein and/or at least one nucleic acid encoding for a urea-cycle protein, typically an mRNA or a pDNA or a viral genome or similar.

In a second aspect the present invention also relates to polypeptide constructs comprising an EV protein fused to a urea cycle protein and/or polypeptide constructs comprising an EV protein (interchangeably called EV enrichment polypeptide or exosomal polypeptide or EV polypeptide or EV protein or similar) fused to a nucleic acid (NA) binding domain, which is used to aid the transport into EVs of a polynucleotide encoding for a UCD protein.

In a third aspect the present invention also relates to a polynucleotide construct encoding for any one of the polypeptide constructs of the present invention, which may be introduced into cells in order to produce EV-producing cells which express one or more of the polypeptide constructs of the present invention.

The present invention also relates to a method of producing EVs according to any one of the preceding claims, comprising: (i) introducing into an EV-producing cell at least one polynucleotide construct according to the invention and (ii) expressing in the EV-producing cell at least one polypeptide construct encoded for by the at least one polynucleotide construct, thereby generating said EVs comprising at least one urea cycle protein, either through direct expression as a UCD protein or via the expression from a polynucleotide (such as an mRNA or any other coding RNA or DNA molecule) that is loaded with the aid of the polypeptide construct.

The present invention also relates to a cell comprising (i) at least one polynucleotide construct according of the invention and/or (ii) at least one polypeptide construct of the invention and/or (iii) at least one EV of the invention.

In a fourth aspect the present invention also relates to a pharmaceutical composition comprising:
(i) at least one polynucleotide construct according to the invention, and/or
(ii) at least one polypeptide construct according to the invention, and/or
(iii) at least one EV according to the invention,
and a pharmaceutically acceptable excipient or carrier; optionally further comprising one or more additional compounds used in the treatment of urea cycle disorders.

The present invention relates to the EVs of the present invention and/or the pharmaceutical composition of the present invention, for use in treating one or more urea cycle disorders. The present invention also relates to the (i) at least one polynucleotide construct according to the invention, (ii) at least one polypeptide construct according to the invention, (iii) at least one EV according to the invention, (iv) at least one cell according to the invention, and/or (v) the pharmaceutical composition according to the invention, for use in medicine, preferably for use in the treatment of one or more urea cycle disorders.

The present invention also relates to a method of treatment of a disease or disorder comprising administering to a patient in need thereof an effective amount of the EV according to the invention.

### BRIEF DESCRIPTION OF THE FIGURES

FIG. 1: Schematic illustration of an EV loaded with an NA cargo molecule, which typically encodes for a UCD protein, using the fusion polypeptide constructs as per the present invention.

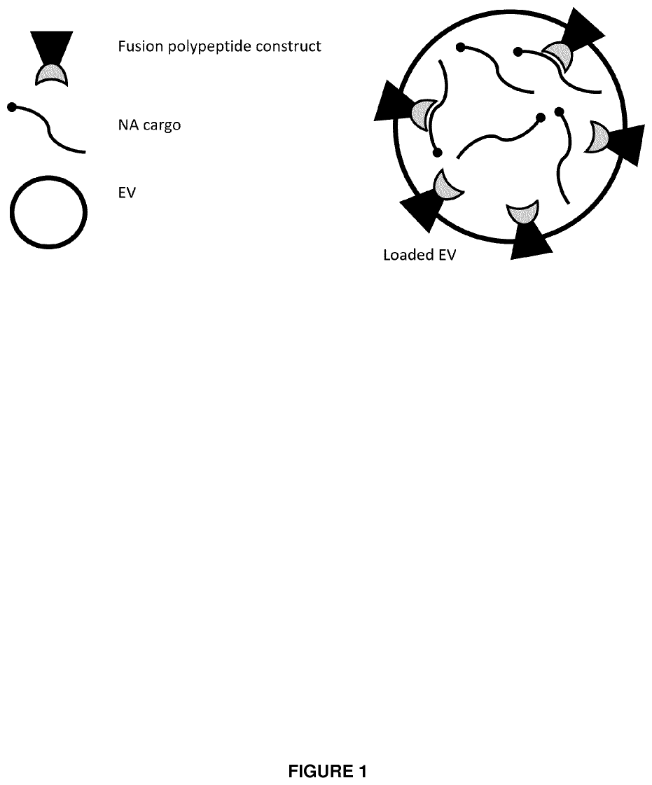

FIG. 2: Schematic illustration of an EV loaded with a UCD protein molecule using the polypeptide loading strategies of as per the present invention.

FIG. 3: Bar chart showing the comparative efficacy of loading a reporter nucleic acid (NanoLuc mRNA) into EVs by an exemplary construct of the present invention (CD63-PUF) compared to the TAMEL loading construct (CD63-MS2). Delivery of reporter mRNA into exosomes is significantly improved by the use of fusion constructs CD63-PUF when compared to control fusion construct CD63-MS2.

FIG. 4: Graph showing delivery in vitro of UCD proteins via EV-mediated protein delivery using different EV engineering approaches. The engineered, modified EVs are able to deliver bioactive UCD proteins at bioactive concentrations.

FIG. 5: Bar chart showing production of fumarate by EVs by ASL engineered exosomes indicating that ALS enzyme loaded into exosomes is catalytically active.

FIG. 6: Chart showing blood ammonia levels of ASL knock-out mice treated with ALS engineered exosomes. The results demonstrate that in-vivo delivery of exosomes engineered to contain urea cycle enzymes is capable of lowering ammonia levels to those of healthy individuals.

### DESCRIPTION OF SEQUENCE LISTINGS

SEQ ID NO 1: Puf 531 protein sequence

SEQ ID NO 2: PUF mRNA loc/PUFeng protein sequence

SEQ ID NO 3: PUF×2 protein sequence

SEQ ID NO 4: Cas6 protein sequence

SEQ ID NO 5: His aptamer protein sequence

SEQ ID NO 6: TAT aptamer protein sequence

SEQ ID NO 7: Human ASL protein sequence

### DETAILED DESCRIPTION OF THE INVENTION

By using inventive EV engineering technology coupled with selective design of protein and polynucleotide cargo molecules, as well as profiling of bioactive EV populations, the present invention addresses several of the key aspects of EV-based therapeutics for UCDs. Importantly, the application of EV-mediated delivery technology for the treatment of UCDs is based on the realization by the inventors of the present invention that EVs, when engineered and modified to comprise therapeutic levels of UCD proteins or the corresponding NAs, constitute a suitable delivery modality for these complex, often hepatocerebral diseases. EVs from select EV-producing cell sources and with particular molecular profiles are especially well suited to drive therapeutic activity in this group of diseases. The non-toxic and non-immunogenic nature of the EVs of the present invention is an important factor for therapeutic activity in vivo in UCDs. Clearly, subjects suffering from hepatic diseases would be unable to tolerate administration of delivery vehicles comprising immuno-toxic bacteriophage proteins and, similarly, other non-EV-based delivery vehicles would be associated with the same issues, namely liver toxicity in patients already having compromised liver function.

For convenience and clarity, certain terms employed herein are collected and described below. Unless otherwise defined, all technical and scientific terms used herein have the same meaning as commonly understood by one of ordinary skill in the art to which this invention belongs.

Where features, aspects, embodiments, or alternatives of the present invention are described in terms of Markush groups, a person skilled in the art will recognize that the invention is also thereby described in terms of any individual member or subgroup of members of the Markush group. The person skilled in the art will further recognize that the invention is also thereby described in terms of any combination of individual members or subgroups of members of Markush groups. Additionally, it should be noted that embodiments and features described in connection with one of aspects and/or embodiments of the present invention also apply mutatis mutandis to all the other aspects and/or embodiments of the invention.

For example, the urea cycle proteins described herein e.g. in connection with the EVs comprising such urea cycle proteins are to be understood to be disclosed, relevant, and compatible with all other aspects, teachings and embodiments herein, for instance aspects and/or embodiments relating to the methods for producing EVs comprising such urea cycle proteins or aspects relating to the polypeptide and/or polynucleotide constructs herein. Furthermore, all polypeptides and proteins identified herein can be freely combined in polypeptide constructs using conventional strategies for fusing polypeptides. As a non-limiting example, all urea cycle proteins described herein may be freely combined in any combination with one or more EV enrichment polypeptides. Also, any and all urea cycle proteins herein may be combined with any other urea cycle protein to generate polypeptide, and/or the corresponding polynucleotide, constructs, comprising more than one urea cycle protein. Moreover, any and all features (for instance any and all members of a Markush group) can be freely combined with any and all other features (for instance any and all members of any other Markush group). Additionally, when teachings herein refer to EVs in singular and/or to EVs as discrete natural nanoparticle-like vesicles it should be understood that all such teachings are equally relevant for and applicable to a plurality of EVs and populations of EVs. As a general remark, the urea cycle proteins, the EV enrichment polypeptides, the tissue targeting moiety, peptides and/or polypeptides, the EV-producing cell sources, and all other aspects, embodiments, and alternatives in accordance with the present invention may be freely combined in any and all possible combinations without deviating from the scope and the gist of the invention.

Furthermore, any polypeptide or polynucleotide or any polypeptide or polynucleotide sequences (amino acid sequences or nucleotide sequences, respectively) of the present invention may deviate considerably from the original polypeptides, polynucleotides and sequences as long as any given molecule retains the ability to carry out the desired technical effect associated therewith. As long as their biological properties are maintained the polypeptide and/or polynucleotide sequences according to the present application may deviate with as much as 50% (calculated using any sequence alignment or sequence homology tool, for instance, BLAST) as compared to the native sequence, although a sequence identity that is as high as possible is preferable (for instance 60%, 70%, 80%, or e.g. 90% or higher). The combination (fusion) of e.g. at least one urea cycle protein with at least one EV enrichment polypeptide naturally implies that certain segments of the respective polypeptides may be replaced and/or modified and/or that the sequences may be interrupted by insertion of other amino acid stretches, meaning that the deviation from the native sequence may be considerable as long as the key properties (e.g. the native effects of the urea cycle proteins, EV trafficking and enrichment, targeting properties, etc.) are conserved. Similar reasoning thus naturally applies to the polynucleotide sequences encoding for such polypeptides. All SEQ ID NOs mentioned herein in connection with peptides, polypeptides and proteins shall only be seen as examples and for information only, and all peptides, polypeptides and proteins shall be given their ordinary meaning as the skilled person would understand them. Thus, as above-mentioned, the skilled person will also understand that the present invention encompasses not merely the specific SEQ ID NOs referred to herein but also variants and derivatives thereof. All proteins, polypeptides, peptides, nucleotides and polynucleotides mentioned herein are to be construed according to their conventional meaning as understood by a skilled person.

The terms “extracellular vesicle” or “EV” or “exosome” are used interchangeably herein and shall be understood to relate to any type of vesicle that is obtainable from a cell in any form, for instance a microvesicle (e.g. any vesicle shed from the plasma membrane of a cell), an exosome (e.g. any vesicle derived from the endo-lysosomal pathway or from any other cellular pathway producing exosomes), an apoptotic body (e.g. obtainable from apoptotic cells), a microparticle (which may be derived from e.g. platelets), an ectosome (derivable from e.g. neutrophils and monocytes in serum), prostatosome (e.g. obtainable from prostate cancer cells), or a cardiosome (e.g. derivable from cardiac cells), etc. Exosomes and/or microvesicles, in particular ARRDC1-mediated microvesicles (ARMMs), represent particularly preferable EVs, but other EVs may also be advantageous in various circumstances.

The EV may be any type of lipid-based structure (with vesicular morphology or with any other type of suitable morphology) that can act as a delivery or transport vehicle. Advantageously, the EV is not an artificial liposome or artificial lipid nano-particle.

The sizes of EVs may vary considerably but an EV typically has a nano-sized hydrodynamic radius, i.e. a radius below 1000 nm. Exosomes often have a size of between 30 and 300 nm, typically between 30 and 200 nm, such as in the range between 50 and 250 nm, which is a highly suitable size range. Clearly, EVs may be derived from any cell type, both in vivo, ex vivo, and in vitro. Preferred EVs of the present invention are exosomes and/or microvesicles but other EVs may also be advantageous in various circumstances. In another preferred embodiment, EVs are preferably obtainable from amnion-derived cells, from Wharton's jelly-derived cells, from amnion epithelial (AE) cells, from mesenchymal stromal cells (MSCs), and from placenta-derived cells. Furthermore, the term “EV” and/or “exosome” and/or “microvesicle” shall also be understood to relate to extracellular vesicle mimics, e.g. cell membrane-based vesicles or EV-based vesicles obtained through for instance membrane extrusion, sonication, or other techniques, etc.

It will be clear to the skilled artisan that when describing medical and scientific uses and applications of the EVs, the present invention normally relates to a plurality of EVs, i.e. a population of EVs which may comprise thousands, millions, billions or even trillions of EVs. As can be seen from the experimental section below, EVs may be present in concentrations such as 10^{5,} 10⁸, 10¹⁰, 10¹¹, 10¹², 10¹³, 10¹⁴, 10¹⁵, 10¹⁸, 10²⁵, 10³⁰ EVs (often termed “particles”) per unit of volume (for instance per ml), or any other number larger, smaller or anywhere in between. In the same vein, the term “population”, which may e.g. relate to an EV comprising a certain urea cycle protein shall be understood to encompass a plurality of entities which together constitute such a population. In other words, individual EVs when present in a plurality constitute an EV population. Thus, naturally, the present invention pertains both to individual EVs and populations comprising EVs, as will be clear to the skilled person. The dosages of EVs when applied in vivo may naturally vary considerably depending on the disease to be treated, the administration route, the activity and effects of the urea cycle protein of interest, any targeting moieties present on the EVs, the pharmaceutical formulation, etc.

The terms “EV enrichment polypeptide”, “EV protein”, “EV polypeptide”, “exosomal polypeptide” and “exosomal protein” are used interchangeably herein and shall be understood to relate to any polypeptide that can be utilized to transport a polypeptide construct (which typically comprises, in addition to the EV enrichment protein, a urea cycle protein or an NA binding domain which binds to an NA cargo molecule encoding for a UCD protein) to a suitable vesicular structure, i.e. to a suitable EV. More specifically, these terms shall be understood as comprising any polypeptide that enables transporting, trafficking or shuttling of a fusion protein construct to a vesicular structure, such as an EV. Examples of such exosomal polypeptides are for instance CD9, CD53, CD63, CD81, CD54, CD50, FLOT1, FLOT2, CD49d, CD71 (also known as the transferrin receptor) and its endosomal sorting domain, i.e. the transferrin receptor endosomal sorting domain, CD133, CD138 (syndecan-1), CD235a, ALIX, AARDC1, palmitoylation signal (Palm), syntenin (also known as syntenin-1), the N terminal portion of syntenin, Lamp2b, syndecan-2, syndecan-3, syndecan-4, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, TSG101, CD34, CD36, CD40, CD40L, CD44, CD45, CD45RA, CD47 (CD47 may be fused at either the alpha, beta or delta positions), CD86, CD110, CD111, CD115, CD117, CD125, CD135, CD184, CD200, CD279, CD273, CD274, CD362, COL6A1, AGRN, EGFR, GAPDH, GLUR2, GLUR3, ARRDC1, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, other exosomal polypeptides, and any fragments, derivatives, domains or combinations thereof, but numerous other polypeptides capable of transporting a polypeptide construct to an EV are comprised within the scope of the present invention. Typically, in many embodiments of the present invention, at least one EV enrichment polypeptide is comprised in the polypeptide construct comprising the urea cycle protein or the NA-binding domains which binds to the NA encoding the UCD protein, and this fusion polypeptide construct may advantageously also comprise various other components, including linkers, transmembrane domains, cytosolic domains, multimerization domains, release domains, etc. Linkers and multimerization domains may be used to advantageously allow the UCD protein or NA binding domain to adopt its proper conformation and therefore either deliver a UCD protein with improved bioactivity or enable improved nucleic acid binding and hence improved nucleic acid loading for hard to load nucleic acids.

The terms “nucleic acid” or “polynucleotide” or “NA” or “NA cargo molecule” or similar are used interchangeably herein and may be used to describe any nucleic acid selected from the group comprising single-stranded RNA or DNA, double-stranded RNA or DNA, and other polynucleotides such as mRNA, plasmids, or any other RNA or DNA vector, such as for instance viral genomes. The NA typically encodes for at least one urea cycle protein but may also encode for other peptides or polypeptides. In several embodiments of the present invention, at least one exosomal polypeptide is fused to an NA-binding domain, in order to form a fusion protein present in an EV for aiding the loading of the NA cargo molecule. Such fusion proteins may also comprise various other components to optimize their function(s), including linkers, transmembrane domains, cytosolic domains, multimerization domains, etc., the advantages of which are described above.

The terms “NA-binding domain” or “NA-binding polypeptide” or “NA-binding protein” are used interchangeably herein and relate to any domain that is capable of binding to a stretch of nucleotides. The NA-binding domains may bind to RNA, DNA, mixmers of RNA and DNA, particular types of NAs such as mRNA, circular RNA or DNA, ribozymes, mini-circle DNA, plasmid DNA, etc. Furthermore, the NA-binding domain(s) may also bind to chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, etc.

Advantageously, the present invention uses NA binding proteins which are all highly conserved amongst eukaryotes and are therefore unlikely to cause adverse immune responses when delivered to patients. Furthermore, the NA-binding domains of the present invention may also bind to either particular sequences of NAs, to domains such as repeats, or to NA motifs, such as stem loops or hairpins. Such binding sites for the NA-binding domains may be naturally occurring in the NA cargo molecule of interest and/or may be engineered into the NA cargo molecule to further enhance EV loading and bioactive delivery. The binding affinity of the NA-binding domain for the nucleic acid is such that the nucleic acid is bound with high enough affinity to be shuttled into the EVs but the affinity of binding is not so high as to prevent the subsequent release of the nucleic acid into the target cell such that the nucleic acid is bioactive once delivered to the target cell. Thus, importantly and in complete contrast to the prior art, the present invention relates to EVs loaded with NA cargo molecules with the aid of releasable NA-binding domains, wherein said NA-binding domains form part of fusion polypeptides with exosomal polypeptides. The NA-binding domains of the present invention have been selected to allow for programmable, modifiable affinity between the NA-binding domain and the NA cargo molecule, enabling production of EVs comprising fusion polypeptides comprising the NA-binding domain and at least one NA cargo molecule, wherein the NA-binding domain of the fusion polypeptide construct interacts in a programmable, reversible, modifiable fashion with the NA cargo molecule, allowing for both loading into EVs and release of the NA cargo molecule either in EVs and/or in or in connection with target cells. This is in complete contrast to the prior art, which merely allows for loading of mRNA molecules into exosomes using the MS2 protein, but wherein the MS2 protein remains bound to the mRNA, inhibiting its release and subsequent translation.

In embodiments of the present invention which utilise NA-binding domains the NA-binding domain may be selected from: PUF proteins, CRISPR-associated polypeptides (Cas) and specifically Cas6 and Cas13, and various types of NA-binding aptamers. The present invention uses the term PUF proteins to encompass all related proteins and domains of such proteins (which may also be termed PUM proteins) from any species, for instance human Pumilio homolog 1 (PUM1), PUM×2 or PUF×2 which are duplicates of PUM1, etc., or any NA-binding domains obtainable from any PUF (PUM) proteins. PUF proteins are typically characterized by the presence of eight consecutive PUF repeats, each of approximately 40 amino acids, often flanked by two related sequences, Csp1 and Csp2. Each repeat has a ‘core consensus’ containing aromatic and basic residues. The entire cluster of PUF repeats is required for RNA binding. Remarkably, this same region also interacts with protein co-regulators, and is sufficient to rescue, to a large extent, the defects of a PUF protein mutant, which makes the PUF proteins highly suitable for mutations used in the present invention. Furthermore, PUF proteins are highly preferred examples of releasable NA-binding domains which bind with suitable affinity to NA cargo molecules, thereby enabling a releasable, reversible attachment of the PUF protein to the NA cargo. PUF proteins are found in most eukaryotes and are involved in embryogenesis and development. PUFs has one domain that binds RNA that is composed of 8 repeats generally containing 36 amino acids, which is the domain typically utilized for RNA binding in this patent application. Each repeat binds a specific nucleotide and it is commonly the amino acid in position 12 and 16 that confer the specificity with a stacking interaction from amino acid 13. The naturally occurring PUFs can bind the nucleotides adenosine, uracil and guanosine, and engineered PUFs can also bind the nucleotide cytosine. Hence the system is modular and the 8-nucleotide sequence that the PUF domain binds to can be changed by switching the binding specificity of the repeat domains. Hence, the PUF proteins as per the present invention can be natural or engineered to bind anywhere in an RNA molecule, or alternatively one can choose PUF proteins with different binding affinities for different sequences and engineer the RNA molecule to contain said sequence. There is furthermore engineered PUF domains that bind 16-nucleotides in a sequence-specific manner, which can also be utilized to increase the specificity for the NA cargo molecule further. Hence the PUF domain can be modified to bind any sequence, with different affinity and sequence length, which make the system highly modular and adaptable for any RNA cargo molecule as per the present invention. PUF proteins and regions and derivatives thereof that may be used as NA-binding domains as per the present invention include the following non-limiting list of PUF proteins: FBF, FBF/PUF-8/PUF-6,-7,-10, all from C. elegans; Pumilio from D. melanogaster; Puf5p/Mpt5p/Uth4p, Puf4p/Ygl014wp/Ygl023p, Puf5p/Mpt5p/Uth4p, Puf5p/Mpt5p/Uth4p, Puf3p, all from S. cerevisiae; PufA from Dictyostelium; human PUM1 (Pumillo 1, sometimes known also as PUF-8R) and any domains thereof, polypeptides comprising NA-binding domains from at least two PUM1, any truncated or modified PUF proteins such as for instance PUF-6R, PUF-9R, PUF-10R, PUF-12R, PUF-16R or derivatives thereof; and X-Puf1 from Xenopus. Particularly suitable NA-binding PUFs as per the present invention includes the following: PUF 531, PUF mRNA loc (sometimes termed PUFengineered or PUFeng), and/or PUF×2, and any derivatives, domains, and/or regions thereof. The PUF/PUM proteins are highly advantageous as they may be selected to be of human origin, which is an advantageous embodiment of the present invention.

Proteins of human origin, rather than those of bacteriophage origin such as the MS2 protein, are beneficial because they are less likely to illicit an adverse immune response. Furthermore, MS2 interacts with a stem loop of bacteriophage origin, which unlike the PUF proteins imply that a prokaryotic NA sequence and motif need to be introduced into the NA molecule of choice. Clearly, this insertion of a stem loop structure of bacteriophage origin and structure may interfere with mRNA translation, resulting in non-functional mRNA cargo molecules, or even trigger immunotoxicity.

Thus, in advantageous embodiments, the present invention relates to eukaryotic NA-binding proteins fused to exosomal proteins. In a preferred embodiment, the NA-binding domain(s) is(are) from the PUF family of proteins, for instance PUF531, PUFengineered, and/or PUF×2, all of which are advantageously of human origin. Importantly, PUF proteins are preferably used in the EV-mediated delivery of mRNA or shRNA, which due to the sequence-specificity of the PUF proteins enables highly controlled and specific loading of the NA drug cargo. In preferred embodiments, the PUF protein(s) are advantageously combined with either transmembrane or soluble exosomal proteins. Advantageous fusion protein constructs include the following non-limiting examples: CD63-PUF531, CD63-PUF×2, CD63-PUFengineered (alternatively known a PUFeng or PUF mRNA loc), CD81-PUF531, CD81-PUF×2, CD81-PUFengineered, CD9-PUF531, CD9-PU×2, CD9-PUFengineered, and other transmembrane-based fusion proteins, preferably based on tetraspanin exosomal proteins fused to one, two or more PUF proteins. Advantageous fusion proteins comprising PUF proteins and at least one soluble exosomal protein include the following non-limiting examples: CD63-PUF, syntenin-PUF531, syntenin-PU×2, syntenin-PUFengineered, syndecan-PUF531, syndecan-PU×2, syndecan-PUFengineered, Alix-PUF531, Alix-PU×2, Alix-PUFengineered, as well as any other soluble exosome protein fused to a PUF protein.

The fact that the PUF proteins have modifiable sequence-specificity for the target NA cargo molecule makes them ideal NA-binding domains for fusing to exosomal polypeptide partner(s). Thus, in preferred embodiments of the present invention, the EVs are loaded with NA cargo molecules using releasable NA-binding domains (as part of fusion proteins with exosomal proteins), wherein the interaction between the NA-binding domain and the NA cargo molecule is advantageously based on specificity for a target nucleotide sequence and not based on a target nucleotide secondary structure (as secondary structures do not enable sequence specificity). In preferred embodiments, the NA cargo molecule is engineered to comprise and/or naturally comprises the target nucleotide sequence for the PUF protein chosen as the NA-binding domain. Such target nucleotide sequences may as abovementioned, for example, be part of the 3′UTR of an mRNA or may be introduced into any NA cargo molecule such as an mRNA, shRNA, miRNA, lncRNA, DNA, etc., allowing for the PUF protein to bind to the NA cargo molecule. The PUF binding site on the NA cargo molecule is typically longer than the sequence bound by many other RNA-binding proteins, such as MS2 which merely recognizes 4 nucleotides and a stem loop in combination, so the preferred stretch of nucleotides on the target binding site may be for instance 5 nucleotides (nt), 6 nt, 7 nt, 8 nt, 9 nt, 10 nt, 11 nt, 12 nt, 13 nt, 14 nt, 15 nt, 16 nt, 17 nt, 18 nt, 19 nt, or even 20 nt and longer, depending on the need for modifiable sequence specificity of the NA-binding domain. In a preferred embodiment, the PUF protein is specific for a natural and/or artificially occurring NA cargo molecule binding site which is 6 nt, 8 nt, 9 nt, 10 nt, 12 nt, or 16 nt in length.

CRISPR-associated polypeptides (Cas) represent another group of NA-binding domains, and may include in particular Cas6 and Cas13 as well as any other RNA binding Cas molecule. Cas6 binds precursor CRISPR RNA (crRNA) with high affinity and process it for later incorporation into for example Cas9. The cleavage rate of the RNA molecule can be modulated and highly defined, hence the association time between the RNA molecule and Cas6 can also be defined in a very accurate fashion, which is important for the purposes of the present invention. Mutant versions of Cas6 or Cas13 may be used which have been mutated to increase or decrease efficiency of RNA cleavage. Mutant versions of Cas6 or Cas13 may be used which have been mutated to increase or decrease the affinity of RNA binding. This will be an advantage for instance when the RNA cargo molecule is to be released in the recipient cell. The defined association time can then be modulated to release the RNA molecule inside the vesicles, but not in the producer cell. The RNA sequence that Cas6 can recognize can be engineered to be inserted into an NA molecule of interest. Cas13 can be engineered to only bind its defined RNA target and not degrade it. By changing the sequence of the sgRNA molecule the Cas13-sgRNA complex can be modulated to bind any RNA sequence between 20-30 nucleotides. Furthermore, as is the case with the PUF proteins, Cas proteins are highly preferred examples of releasable NA-binding domains which bind with suitable affinity to NA cargo molecules, thereby enabling a releasable, reversible attachment of the Cas protein to the NA cargo. As with the PUF-based NA-binding domains, the Cas proteins represent a releasable, irreversible NA-binding domain with programmable, modifiable sequence specificity for the target NA cargo molecule, enabling higher specificity at a lower total affinity, thereby allowing for both loading of the NA cargo into EVs and release of the NA cargo in a target location.

NA aptamer-binding domains are another group of NA-binding domains as per the present invention. Such NA aptamer-binding domains are domains, regions, stretches of amino acids, or entire polypeptides or proteins that can be bound with specificity by NA-based aptamers. Aptamers are RNA sequences that form secondary and/or tertiary structures to recognize molecules, similar to the affinity of an antibody for its target antigen. Hence these RNA molecules can recognize specific amino acid sequences with high affinity. RNA aptamers are applied in the present invention by inserting particular nucleotide sequences into the NA molecule to recognize specific amino acid sequences. Such amino acid sequences can be engineered into and/or next to the exosomal carrier polypeptide to enable the aptamer (which is engineered into and/or next to the NA cargo molecule) to bind to it, thereby shuttling the NA cargo molecule into EVs with the aid of the exosomal polypeptide. Two aptamers with suitable characteristics are a His-aptamer with high affinity for a stretch of histidine (His) amino acids and an aptamer towards the HIV Tat domain. The aptamer sequence(s) are preferably inserted in the 3′ and/or 5′ untranslated region of an mRNA or unspecific region of non-coding RNAs. Two or more aptamers can also be combined into one NA cargo molecule to increase the specificity and avidity to the exosomal carrier protein. Importantly, all the NA-binding domains of the present invention provide for programmable, sequence-specific, reversible, releasable binding to the NA cargo molecule, for instance mRNA, which is in complete contrast to the high-affinity, irreversible binding to RNA found in the prior art. In preferred embodiments of the present invention, the NA-binding domains are either PUF proteins or Cas proteins, due to their easily programmable nature and sequence specificity combined with their reversible, releasable binding to NA cargo molecules. Importantly, the sequence specificity of Cas proteins and PUF proteins as NA-binding domains is preferably based on interaction with at least 6 nt, preferably at least 8 nt on the target NA molecule, which when combined with a low-affinity interaction allows for high productive EV-mediated delivery of the NA cargo molecule. The at least 6 nt binding site on the NA cargo molecule is preferably present in a contiguous sequence of nucleotides. The binding site of the NA cargo molecule thus preferably corresponds in length to two codons.

The terms “UCD protein” or “urea cycle protein” or similar are used interchangeably herein and shall be understood to relate to any polypeptide belonging to the group of urea cycle proteins, i.e. enzymes and other proteins that form part of participate in the urea cycle. Non-limiting examples of UCD proteins include N-acetylglutamate synthase, carbamoyl phosphate synthetase, ornithine transcarbamoylase, carbamyl phosphate synthetase, argininosuccinic acid synthase, argininosuccinate synthetase, argininosuccinic acid lyase (also known as argininosuccinate lyase), arginase, mitochondrial ornithine transporter, ornithine translocase, citrin, etc.

The present invention relates to extracellular vesicles (EVs) comprising at least one fusion polypeptide comprising at least one nucleic acid (NA)-binding domain and at least one exosomal polypeptide, wherein the at least one NA-binding domain may be one or more of PUF, a CRISPR-associated (Cas) polypeptide, and/or an NA aptamer-binding domain. As a result of presence of the NA-binding domain, the EVs typically further comprise at least one NA cargo molecule, which typically encodes for a UCD protein. Normally, the number of NA cargo molecules that are comprised in each and every EV is considerable, which is a clear improvement over the prior art which normally achieves a very low loading efficacy and highly variable loading across a given population of EVs. In the case of the present invention, the inventive design of the fusion polypeptide constructs means that the at least one NA cargo molecule is very efficiently transported into the EV (with the help of the fusion polypeptide) followed by a significantly improved release process. The releasable nature of the binding between the NA-binding domain (which is comprised in the fusion polypeptide) and the NA cargo molecule is a key aspect of the present invention, as it allows for binding of NA cargo molecules in the EV-producing cells (where NA cargo molecules are normally overexpressed) while enabling delivery of bioactive NA molecules in and/or near the target cell.

Thus, unlike in the prior art, a programmable, lower affinity interaction between the NA-binding domain and the NA cargo molecules enables the present invention to efficiently load EVs in EV-producing cells, whilst also enabling release of NA cargo in suitable locations (typically inside a target cell) where the lower affinity and the releasable nature of the interaction between the NA cargo molecule and the NA-binding domain is highly advantageous. Furthermore, unlike the prior art which merely discloses MS2 as a high-affinity RNA-binding protein binding to 4 nts and a stem loop, the present invention allows for sequence-specific low-affinity or medium-affinity binding to stretches of nucleotides that are longer and thereby more specific, for instance 6 nt in length, or 8 nt in length.

The longer length of binding site enables a range of different mutations to be introduced which generate binding sites with a range of modified binding affinities, thus producing the programmable lower affinity interactions mentioned above. For instance, introduction of a single point mutation into a 6 or 8 nucleotide region will subtly modify the binding affinity, whereas, even a single mutation in the shorter 4 nucleotide binding region of MS2 is known to significantly affect the binding affinity of MS2 for the RNA. The longer length of nucleic acid provides more scope to introduce one or more mutations which affect the binding affinity of the protein for the nucleic acid. Similarly, requiring a longer stretch of nucleotides to be bound results in a larger number of amino acids which are capable of interacting with the longer nucleotide sequence and thus providing more possibilities for mutating those interacting amino acids and again producing a larger range of possible protein mutants with a variety of binding affinities. Both the longer nucleotide binding site and the larger protein binding sites of PUF, Cas6 and Cas13 provide advantages in enabling a greater range of affinities to be achieved by mutation than could be achieved by mutation of the MS2 protein or the MS2 RNA sequence. Thus, this longer sequence affords greater possibilities to engineer the nucleic acid and/or the binding protein to tailor the binding affinity specifically to an individual cargo of interest if needed to improve the release of that cargo nucleic acid. As has been discussed above, the ability to control the affinity of binding to the nucleotide cargo and thus modify and control the releasability of the nucleotide cargo is a significant advantage of the present invention over the prior art resulting in delivery and release of bioactive nucleic acids. Importantly, as abovementioned, the immune-toxicity stimulated by bacteriophage proteins would be particularly problematic in the case of disease involving the liver, as the EV biodistribution pattern would result in substantial accumulation of the bacteriophage proteins in the liver, thereby driving an increased negative impact on liver function in patients already suffering from a compromised hepatic system.

In a further embodiment, the EVs may further comprise an organ, tissue or cell targeting peptide and/or polypeptide. An example of a targeting peptide which has proven potent in transporting EVs into the brain and the CNS, which may be important in certain UCDs that have CNS manifestations, is the rabies virus glycoprotein (RVG) peptide, but other peptides and polypeptides are also within the scope of the present invention. Importantly, the tissue targeting peptide and/or polypeptide may be comprised in the polypeptide construct which also comprises the urea cycle polypeptide (and optionally the EV enrichment polypeptide for enhanced loading of the UCD protein and/or the corresponding coding NA cargo molecule) and/or may be present as a separate polypeptide construct in the EVs. When the targeting peptide and/or polypeptide is part of a separate polypeptide construct it is preferably fused to an exosomal protein, to ensure efficient loading into the EVs.

When the EVs as per the present invention comprise at least one targeting moiety, said targeting moiety is able to target the EV plus the associated polypeptide or polynucleotide cargo for targeted delivery to a cell, tissue, organ, and/or compartment of interest. The targeting moiety may be comprised in the fusion polypeptide itself, which is especially advantageous when using an exosomal polypeptide with a transmembrane domain to enable display of the targeting moiety on the surface of the EVs. Targeting moieties may be proteins, peptides, antibodies, nanobodies, alphabodies, single chain fragments or any other derivatives of antibodies or binders, etc. The targeting moiety may also form part of a separate polypeptide construct which is comprised in the EV. Further, the fusion polypeptides comprised in the EVs of the present invention may also comprise various additional moieties to enhance bioactive delivery. Such moieties and/or domains may include the following non-limiting examples of functional domains: (i) multimerization domains which dimerize, trimerize, or multimerize the fusion polypeptides to enhance EV formation and/or improve loading, (ii) linkers, as above-mentioned, to avoid steric hindrance and provide flexibility, between e.g. the UCD protein and the exosomal protein or between an exosomal protein and an NA-binding domain, (iii) release domains, such as cis-cleaving elements like inteins, which have self-cleaving activity which is useful for release of particular parts of the fusion polypeptide (for instance releasing the UCD protein and/or the NA cargo which encodes the UCD protein), (iv) RNA cleaving domains for improved release of the RNA in recipient cells, for instance domains encoding for nucleases such as Cas6, Cas13, (v) endosomal escape domains, such as HA2, VSVG, GALA, B18, etc., and/or (vi) nuclear localization signals (NLSs). The tissue targeting moiety may be a tissue targeting peptide and/or polypeptide which may be comprised in the same polypeptide construct as the therapeutic peptide and/or is present as a separate polypeptide construct.

In one embodiment, the NA cargo molecule may be selected from the group comprising mRNA, circular RNA, mini-circle DNA, plasmid DNA, or a viral genome, but essentially any type of NA molecule can be comprised in the EVs as per the present invention, as long as it can encode for the UCD proteins that need to be replaced in the UCDs. Both single-stranded and double-stranded NA molecules are within the scope of the present invention, and the NA molecule may be naturally occurring (such as RNA or DNA) or may be a chemically synthesized RNA and/or DNA molecule which may comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, etc. Importantly, although the present invention is highly suitable for endogenous loading of NA cargo molecules (for instance mRNA, circular RNA, viral genomes, etc.) it is also applicable to loading with exogenous NA molecules which may be loaded by exposing EV-producing cells to the NA molecule in question and/or by co-incubation or formulating the NA cargo molecule with the EVs per se.

The NA cargo molecule may be linear, circularized, and/or have any secondary and/or tertiary and/or other structure. The NA cargo molecule may comprise one or more of the following: (i) a site for mi RNA binding, wherein such site optionally is tissue and/or cell type specific; (ii) at least one stabilizing domain, such as a polyA tail or a stem loop; or, (iii) at least one hybrid UTR in the 5′ and/or 3′ end.

In embodiments of the present invention, the NA cargo molecules as per the present invention comprise (i) at least one binding site for the NA-binding domain of the fusion polypeptide and (ii) a polynucleotide domain encoding for the therapeutic UCD protein(s). In preferred embodiments, the NA cargo molecules comprise at least two binding sites and even more preferably a higher number of binding sites, e.g. 3, 4, 5, 6, 7, 8, 9, 10, 15, or an even greater number. The inventors have realized that including 1-8 binding sites yields optimal loading of the NA cargo molecule into EVs without negatively impacting the release and bioactive delivery of the cargo. The binding sites for the NA-binding domain, can be genetically engineered into and/or flanking the 3′ and/or 5′ UTR and/or by sequence optimization be placed in the coding region of the NA cargo molecule.

The designs of both the NA cargo molecule (i.e. the polynucleotide encoding the UCD protein in question) and the fusion polypeptide constructs which include NA-binding domains are key to loading, release, and bioactive delivery, e.g. into target cells and/or into particular organs, tissues, and bodily compartments. As abovementioned, the NA-binding domains utilized in the present invention are highly advantageous as they avoid triggering immune-stimulation and toxicity, which is especially important as the EVs are meant to deliver UCD proteins and/or the corresponding NA cargo to the liver, which is already comprised by the impact of the urea cycle disorders per se. The inventors have discovered that particularly advantageous embodiments are EVs comprising fusion polypeptides which comprises at least one exosomal polypeptide flanked on both sides by at least one NA-binding domain (i.e. at least one NA-binding domain on each side). Alternatively, the NA-binding domain may in various instances by inserted into the exosomal polypeptide in at least one location (for instance on a extravesicular loop of e.g. CD63), for instance when it is desirable to display the NA-binding domain on the outside of the EV to enhance exogenous loading. The exosomal polypeptide may be flanked immediately C and/or N terminally, but one of the most advantageous designs is to include a linker peptide and/or a cleaving polypeptide domain (such as an intein) between the exosomal polypeptide and the NA-binding domains (or in the case of protein delivery release of the UCD protein as such), to provide spacing and flexibility for maintained activity of both the exosomal polypeptide(s) and the NA-binding domain(s). Such linkers may advantageously be glycine-serine (GS) linkers containing a particular number of repeats. The inventors have realized that either 1 to 4 repeats are the most advantageous, providing enough flexibility without rendering the fusion polypeptide too unstructured. As above-mentioned, for applications involving exogenous loading of NA cargo molecules, EVs preferably comprise fusion polypeptides which comprises at least one exosomal polypeptide fused to at least one NA binding domain on its N terminal, and/or its C terminal and/or in any extravesicular (i.e. present outside of the EV) regions of the exosomal polypeptide, in order to expose the NA binding domain on the surface of exosome.

The present invention also relates to various inventive modifications of the NA cargo molecule, which are key to ensure high efficiency of loading, release and bioactive delivery. For instance, by designing the NA cargo molecule to be either linear or circular one can increase or decrease aspects such as loading efficiency and stability. Furthermore, by optimizing the design of the sequence it is also possible to influence secondary and tertiary structures of the NA cargo, which can further facilitate loading, by facilitating the easy accessibility of NA binding domain to the target NA.

In yet another advantageous embodiment, the NA cargo molecule may comprise additional moieties to increase potency, either by enhancing loading, improving release, increasing tissue-specific activity, and/or increase the stability of the NA cargo molecule. For instance, the NA cargo molecule may comprise one or more of the following: (i) a site for miRNA binding, wherein such site optionally is tissue and/or cell type specific, to drive preferential cell and/or tissue specific activity, (ii) at least one stabilizing domain, such as a long PolyA tail or more than one PolyA tail (for instance 2 or 3 or even 4 PolyA tails), (iii) at least one stem loop structure in the 5′ and/or 3′ UTR, in order to inhibit nuclease degradation, (iv) an RNA polymerase to drive transcription of the NA cargo molecule, (v) codon-optimized sequences to increase mRNA stability, (vi) at least one hybrid UTR in the 5′ and/or 3′ end to increase mRNA translation efficiency, and/or (vii) ribozyme(s).

As abovementioned, the NA cargo molecule (i.e. the polynucleotide encoding for the UCD protein) may advantageously comprise (i) at least one binding site for the NA-binding domain for colocalization into the EVs and (ii) a polynucleotide domain encoding the therapeutic UCD protein. The NA cargo molecule may advantageously further comprise a cleavage site between the at least one binding site and the coding NA component. The fusion polypeptide comprising the NA-binding domains may comprise at least one exosomal polypeptide flanked N- and/or C terminally by NA-binding domains and/or wherein the at least one NA-binding domain is inserted into the EV polypeptide sequence.

The EVs as per the present invention are loaded with the NA cargo molecule with the aid of the fusion polypeptide, which normally comprises an exosomal polypeptide fused to at least one NA-binding domain which binds to the NA cargo molecule and transports it into the EVs. Without wishing to be bound by any theory, it is surmised that the loading takes place in connection with the formation of the EV inside the EV-producing cell or exogenously by incubating NA cargo molecule(s) with engineered EVs. The fusion polypeptide may normally bind to the NA cargo molecule (such as an mRNA molecule co-expressed in the EV-producing cell) and transport it into the vesicle which is then secreted from the producer cell as an EV. As mentioned, the NA cargo molecule may be expressed in the same EV-producing cell as the fusion polypeptide (endogenous loading) and/or it may be loaded exogenously into an EV once the EV is formed and optionally purified. Co-expression in the EV-producing cell of the NA cargo is a highly advantageous embodiment, as the EV production takes place in a single step in a single cell, which enables scaling the process and simplifies both upstream and downstream processing. The NA cargo molecule (e.g. an mRNA or any other UCD protein-coding NA molecule, etc.) may be expressed from the same polynucleotide constructs as the fusion polypeptide, or it may be expressed from another polynucleotide construct. Both methods have advantages: the use of one construct ensure that both the fusion polypeptide and the NA cargo molecule are translated/transcribed together whereas the use of more than one construct enables differential expression of the two components, e.g. a higher expression level of either the fusion polypeptide or the NA cargo molecule. In preferred embodiments, the polynucleotide construct(s) from which the fusion polypeptide and/or the NA cargo molecule is/are expressed is advantageously stably introduced into the EV-producing cells, to enable consistent, reproducible and high-yield production of the NA-loaded EVs. Creation of stable cells, preferably followed by single cell cloning to obtain a single cell clone for EV production, is equally important for loading of coding NA molecules as for loading of fusion polypeptides comprising UCD proteins into EVs. In a preferred embodiment, the EV-producing cells are stably transfected and/or transduced with bicistronic or multicistronic vectors (also known as constructs or polynucleotides, etc.) comprising the fusion polypeptide and the NA cargo molecule. Such bicistronic or multicistronic construct may comprise e.g. inducible promoters, IRES element(s) or 2A peptide linkages, allowing for the expression of both (i) the fusion polypeptide comprising the NA-binding domain and the exosomal protein, and (ii) the NA cargo molecule of interest, for instance an mRNA or any other type of coding NA cargo molecule. In addition to using bicistronic or multicistronic vectors, multiple or bidirectional promoters represent another tractable method for stably inserting a single construct encoding for the two components of interest that are to be loaded into the EVs according to the present invention. Clearly, in alternative embodiments, two or more constructs (for instance plasmids) may also be transfected and/or transduced into EV-producing cells, although the use of single constructs may be advantageous as it may enable equimolar concentrations of the fusion polypeptide (and thus the NA-binding domain) and the NA cargo molecule per se. Importantly, the EV-producing cells of the present invention are normally designed to overexpress the at least one polynucleotide construct, which allows for appropriate production of the NA cargo molecule at a suitable concentration in the EV-producing cell, thereby allowing for the reversible, releasable attachment of the NA-binding domain to the NA molecule. Overexpression of the polynucleotide(s) is an important tool that allows for creating a relatively high UCD protein fusion polypeptide or UCD protein-coding NA cargo molecule concentration in the EV-producing cell, while allowing at the same time for release of the NA cargo molecule in the target cell where the concentration of the NA cargo molecule is lower. This is especially relevant for the PUF and Cas proteins.

As above-mentioned, EVs are typically present not as single vesicles but in a substantial plurality of vesicles, and the present invention hence also relates to populations of EVs. In advantageous embodiments, the average number of NA cargo molecules per EV throughout such a population is above on average one (1) NA cargo molecule per EV, preferably above 10 NA cargo molecule per EV, and even more preferably above 100 NA cargo molecule per EV. However, throughout the population there may also be EVs which do not comprise any NA cargo molecules, and the present invention may thus also relate to populations of EVs which comprise on average less than one (1) NA cargo molecule per EV.

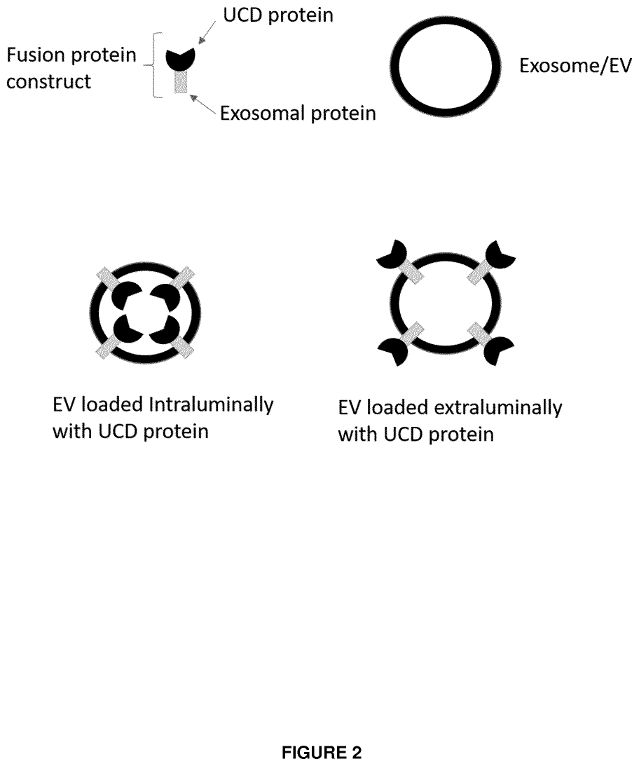

Importantly, the prior art typically merely yields loading of the RNA cargo into a small fraction of the EVs, in a very inefficient manner. For instance, the TAMEL system results in virtually zero to sub-single percentage loading of single EVs. The inventors of the TAMEL system reports that loading of an RNA molecule into exosomes is enhanced when using the TAMEL system at most 7-fold, whereas the present invention improves productive loading of e.g. mRNA and other NA cargo molecules by typically at least 10-fold, preferably at least 25-fold, but frequently by at least 50-fold, and preferably by at least 70-fold, as compared to (i) EVs without NA-binding domain present in the fusion protein and/or without binding site for the NA-binding domain in the NA cargo molecule, (ii) EVs without the fusion protein per se (for instance as shown in FIG. 2), (iii) un-engineered EVs which are only passively loaded with the NA cargo molecule, and/or (iv) any given internal NA control molecule. Thus, the present invention provides for a way of loading considerably more NA cargo molecules into a given population of EVs, and importantly the present invention also enables loading a significantly higher proportion of EVs as compared to the prior art. In one embodiment, the present invention relates to EV populations wherein at least 5%, at least 10%, at least 20%, at least 50%, at least 70%, at least 75%, at least 80%, at least 85%, at least 90%, and/or at least 95% of all EVs comprise an NA cargo molecule in question. As abovementioned, a crucial difference between the present invention and, for instance, U.S. Ser. No. 14/502,494 and other prior art documents relate to the ineffective and importantly uneven distribution of fusion polypeptides across whole populations of EVs. The MS2 protein used in U.S. Ser. No. 14/502,494, for instance, is only present in a small fraction of EVs, which results in unevenly distributed loading of mRNA cargo across EV populations. Conversely, the fusion proteins of the present invention are evenly distributed across entire EV populations, which means that essentially each and every EV comprises at least one fusion polypeptide as per the present invention and normally at least one NA cargo molecule. Thus, in one embodiment, the present invention relates to an EV composition comprising essentially two EV subpopulations, wherein (i) the first EV subpopulation comprises on average more than one fusion polypeptide (comprising the NA-binding domain and the exosomal polypeptide) per EV, and (ii) wherein the second EV subpopulation comprises the NA cargo molecule in question combined with on average more than one fusion polypeptide per EV. In contrast, the prior art, for instance U.S. Ser. No. 14/502,494, teaches EVs which comprise very few fusion polypeptides per EV, typically less than 1 fusion polypeptide per 10 EVs, which clearly implies that the productive loading and delivery of an NA cargo molecule that is dependent on said fusion protein will be significantly lower than is the case in the present application. Without wishing to be bound by any theory, it is surmised that the reason for the prior art's failure to achieve higher loading of the fusion protein into EVs results from the fact that MS2 and similar non-eukaryotic proteins do not shuttle efficiently into exosomes and/or that they trigger toxicity, two issues that are addressed by the present invention.

The EVs of the present invention when loaded with UCD protein may comprise at least one copy of the polypeptide construct (i.e. the UCD protein, optionally fused to an exosomal protein) per EV. More preferably a single EV of the present invention may comprise: (i) at least 10 copies of the polypeptide construct; (ii) at least 50 copies of the polypeptide construct; and/or (iii) at least 100 copies of the polypeptide construct.

The polypeptide constructs of the present invention comprise at least one therapeutic urea cycle protein combined in one polypeptide construct with at least one EV enrichment polypeptide, for instance CD63, CD81, CD9, syntenin, Lamp2B, Lamp2A, syndecan, Alix, CD47, palmitoylation domain(s), myristoylation domain(s), or any other EV enrichment polypeptide which can be operably linked to the therapeutic urea cycle protein on both a polynucleotide and a polypeptide level.

As abovementioned, the polypeptide construct(s) comprised in the EVs as per the present invention may in advantageous embodiments be engineered to comprise at least one EV enrichment polypeptide, in order to drive the internalization into EVs of the urea cycle proteins. Such EV enrichment polypeptides may be selected from essentially any EV polypeptide, for instance from the following group of EV enrichment polypeptides: CD9, CD53, CD63, CD81, CD54, CD50, FLOT1, FLOT2, CD49d, CD71, CD133, CD138, CD235a, ALIX, Syntenin-1, Syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, ARRDC1, AGRN, EGFR, GAPDH, GLUR2, GLUR3, palmitoylation domain, myristoylation domain, HLA-DM, HSPG2, Hsp70, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, any derivatives and/or domains thereof, and any fragments, derivatives, domains or combinations thereof. Any UCD protein may be combined in a fusion protein with any EV enrichment polypeptide of the present invention. Further advantageously the polypeptide constructs of the present invention may further comprise an intein which enables the UCD protein cargo to be cleaved and therefore released from the EV enrichment polypeptide by the self-cleaving activity of the intein.

In further embodiments of the present invention, the urea cycle protein or NA molecule coding for such UCD proteins is selected from the group comprising N-acetylglutamate synthase, carbamoyl phosphate synthetase, ornithine transcarbamoylase, argininosuccinic acid synthase, argininosuccinate synthetase, argininosuccinic acid lyase, arginase, mitochondrial ornithine transporter, ornithine translocase, citrin, y+L amino acid transporter 1, uridine monophosphate synthase or any fragments, derivatives, domains or combinations thereof.

As described above, in another aspect, the present invention relates to inventive fusion polypeptides comprising at least one NA-binding domain and at least one exosomal polypeptide, wherein the at least one NA-binding domain is one or more of PUF, Cas, and/or an NA aptamer-binding domain. In advantageous embodiments, the fusion polypeptides may optionally further comprise additional regions, domains, sequences, and/or moieties endowing the polypeptide with particular functions. Non-limiting examples of additional domains comprised in the fusion polypeptide include (i) multimerization domains, (ii) linkers, (iii) release domains, (iv) RNA cleaving domains, (v) endosomal escape moieties, (vi) protease specific cleavage sites, (vii) inteins (viii) targeting moieties and/or (ix) self-cleaving domains such as inteins.

Multimerization domains enable dimerization, trimerization, or any higher order of multimerization of the fusion polypeptides, which increases the sorting and trafficking of the fusion polypeptides into EVs and may also contribute to increase the yield of vesicles produced by EV-producing cells. Linkers are useful in providing increased flexibility to the fusion polypeptide constructs, and also to the corresponding polynucleotide constructs, and may also be used to ensure avoidance of steric hindrance and maintained functionality of the fusion polypeptides. Release domains may be included in the fusion polypeptide constructs in order to enable release of particular parts or domains from the original fusion polypeptide. This is particularly advantageous when the release of parts of the fusion polypeptide would increase bioactive delivery of the NA cargo and/or when a particular function of the fusion polypeptide works better when part of a smaller construct. Suitable release domains may be cis-cleaving sequences such as inteins, light induced monomeric or dimeric release domains such as Kaede, KikGR, EosFP, tdEosFP, mEos2, PSmOrange, the GFP-like Dendra proteins Dendra and Dendra2, CRY2-CIBN, etc. NA-cleaving domains may advantageously also be included in the fusion polypeptides, to trigger cleave of the NA cargo. Non-limiting examples of NA cleaving domains include endonucleases such as Cas6, Cas13, engineered PUF nucleases, site specific RNA nucleases etc. Furthermore, the fusion polypeptides of the present invention may also include endosomal escape domains to drive endosomal escape and thereby enhance the bioactive delivery of the EV per se and the EV NA cargo molecule. Another strategy for enhancing delivery is to target the EVs to cells, tissues, and/or organs or other bodily compartments. Targeting can be achieved by a variety of means, for instance the use of targeting peptides. Such targeting peptides may be anywhere from a few amino acids in length to 100s of amino acids in length, e.g. anywhere in the interval of 3-100 amino acids, 3-30 amino acids, 5-25 amino acids, e.g. 7 amino acids, 12 amino acids, 20 amino acids, etc. Targeting peptides of the present invention may also include full length proteins such as receptors, receptor ligands, etc. Furthermore, the targeting peptides as per the present invention may also include antibodies and antibody derivatives, e.g. monoclonal antibodies, single chain variable fragments (scFvs), other antibody domains, etc.

In particularly preferred embodiments of the present invention the polypeptide constructs comprise ASL protein displayed extraluminally using a fusion protein comprising LAMP2b-ASL; CD47alpha-ASL; CD47beta-ASL; CD47delta-ASL and/or CD47gamma-ASL (CD47alpha/beta/gamma/delta represent successively more truncated versions of the CD47 protein). Alternatively, in other equally preferred embodiments of the present invention the polypeptide constructs comprise ASL protein displayed intraluminally using a fusion protein comprising CD63-Intein-ASL and/or Palm-Intein-ASL (Palm being a palmitoylation sequence). The EVs of the present invention may also comprise combinations of any or all of these extra and intraluminally displaying protein constructs. Furthermore, the ASL protein in the preferred embodiments may be replaced by any other urea cycle protein. The benefit of intraluminal loading of the urea cycle protein/polynucleotide encoding the urea cycle protein is that the protein/polynucleotide will be protected against degradation by encapsulation inside the EV, thereby extending the half-life of the cargo molecule.

Using a palmitoylation sequence is particularly advantageous because palmitoylation is a reversible process which allows proteins to dynamically re-localize between the cytosol and intracellular/plasma membranes. The effect of this is twofold: firstly in EV producer cells it allows the polypeptide construct of the present invention to be recycled within the producer cell such that if the polypeptide initially locates to a membrane that does not produce EVs it can subsequently re-localise to different subcellular membrane which is capable of producing EVs and therefore increase the levels of produced polypeptide construct which are eventually incorporated into EVs; secondly once the EV is delivered to target cells the fatty acid can be removed by depalmitoylation enzymes and thus the cargo can be delivered in a free un-attached form (without necessarily needing an intein or other cleavage mechanism to be built into it) allowing the delivered cargo protein to obtain its optimal bioactive confirmation and thus display enhanced therapeutic effects. Utilising palmitoylation therefore has a surprising and unexpected dual role of acting to locate the cargo to the EV during the upstream processing of the EV-producing cells as well as enabling the release of the cargo in the relevant target location.

In yet another aspect, the present invention relates to polynucleotide constructs encoding for the polypeptide constructs as per the present invention. Such polynucleotide construct may naturally be expressed in vivo, ex vivo, and/or in vitro, using various vectors. Suitable vectors comprising the polynucleotide constructs as per the present invention include, in yet another aspect: plasmids; mini-circles; any type of substantially circularized polynucleotide; viruses such as adenoviruses, adeno-associated viruses, lentiviruses, and/or capsid-free viruses and/or viral genomes; linear DNA and/or RNA polynucleotides; native messenger RNAs (mRNAs); and/or modified mRNAs, which typically comprise modified nucleosides, such as 5-methylcytidine and pseudouridine, to reduce immunogenicity and enhance mRNA stability.

The polynucleotide constructs as per the present invention may further comprise one or more sites or domains for imparting particular functionality into the polynucleotide. For example, the stability of the polynucleotide constructs can be enhanced through the use of stabilizing domains, such as polyA tails or stem loops, and the polynucleotide construct may also be controlled by particular promotors which may optionally be cell-type specific, inducible promotors, linkers, etc. A PolyA tail may also be inserted upstream of the Cas6 or Cas13 cut site of the mRNA cargo molecule so as to result in cleavage of mRNAs which retain the stabilizing PolyA tail. This has the benefit that the cargo mRNA has increased stability in-vivo allowing resulting in more protein being translated from a single cargo mRNA and thus greater therapeutic bioactivity delivered per EV.

The present invention also relates to cells comprising (i) at least one polynucleotide construct according of the invention and/or (ii) at least one polypeptide construct of the invention and/or (iii) at least one EV of the invention.

The terms “source cell” or “EV source cell” or “parental cell” or “cell source” or “EV-producing cell” or any other similar terminology shall be understood to relate to any type of cell that is capable of producing EVs under suitable conditions, typically in cell culture. Cell culture may include suspension culture, adherent culture or any other type of culturing system, in vivo, ex vivo and/or in vitro. Source cells as per the present invention may also include cells producing exosomes in vivo, e.g. via delivery of a polynucleotide construct into a subject for subsequent translation and in vivo production of EVs, in e.g. the liver.

Generally, EVs may be derived from essentially any cell source, be it a primary cell source or an immortalized cell line. The EV source cells may be any embryonic, fetal, and adult somatic stem cell types, including induced pluripotent stem cells (iPSCs) and other stem cells derived by any method, as well as any adult cell source. The source cells per the present invention may be select from a wide range of cells and cell lines, for instance mesenchymal stem or stromal cells (obtainable from e.g. bone marrow, adipose tissue, Wharton's jelly, perinatal tissue, chorion, placenta, tooth buds, umbilical cord blood, skin tissue, etc.), fibroblasts, amnion cells and more specifically amnion epithelial cells optionally expressing various early markers, myeloid suppressor cells, M2 polarized macrophages, adipocytes, endothelial cells, fibroblasts, etc. Cell lines of particular interest include human umbilical cord endothelial cells (HUVECs), human embryonic kidney (HEK) cells, endothelial cell lines such as microvascular or lymphatic endothelial cells, erythrocytes, erythroid progenitors, chondrocytes, MSCs of different origin, amnion cells, amnion epithelial (AE) cells, any cells obtained through amniocentesis or from the placenta, airway or alveolar epithelial cells, fibroblasts, endothelial cells, etc. Also, immune cells such as B cells, T cells, NK cells, macrophages, monocytes, dendritic cells (DCs) are also within the scope of the present invention, and essentially any type of cell which is capable of producing EVs is also encompassed herein.

When treating neurological diseases, one may contemplate to utilize as source cells e.g. primary neurons, astrocytes, oligodendrocytes, microglia, and neural progenitor cells. The source cell may be either allogeneic, autologous, or even xenogeneic in nature to the patient to be treated, i.e. the cells may be from the patient himself or from an unrelated, matched or unmatched donor. In certain contexts, allogeneic cells may be preferable from a medical standpoint, as they could provide immuno-modulatory effects that may not be obtainable from autologous cells of a patient suffering from a certain indication. For instance, in the context of treating systemic, peripheral and/or neurological inflammation, allogeneic MSCs or AEs may be preferable as EVs obtainable from such cells may enable immuno-modulation via e.g. macrophage and/or neutrophil phenotypic switching (from pro-inflammatory M1 or N1 phenotypes to anti-inflammatory M2 or N2 phenotypes, respectively). The most advantageous source cells per the present invention are MSCs, amnion-derived cells, amnion epithelial (AE) cells, any perinatal cells, and/or placenta-derived cells, all of which are of mammal, most preferably of human, origin. The cell lines from which EVs are derived may be adherent or suspension cells and may be generated as stable cell lines or single clones.

In one embodiment the present invention relates to EVs obtainable from MSCs, AE cells or placenta-derived cells, so called MSC-EVs, AE-EVs, and P-EVs. Such cells are particular preferable as they appear to allow for the production of EVs as per the present invention which comprise a significant number of copies, i.e. a considerable plurality, of polypeptide constructs comprising at least one urea cycle protein, in order to enhance their therapeutic activity in various different UCDs. The term “endogenously engineered” means that EV-producing cells are genetically engineered to contain a polynucleotide construct which encodes for a therapeutic urea cycle protein, which is incorporated into the EVs with the aid of the cellular machinery. Although the abovementioned cell sources are preferable embodiments the present invention relates to any EV-producing cell source, i.e. any cell that can produce EVs. The abovementioned cell sources are also highly efficient at producing EVs comprising NA cargo molecules encoding for bioactive UCD proteins.

MSC-EVs, AE-EVs, and P-EVs and various other EV-producing cell sources are unexpectedly capable of carrying large number of copies of correctly folded UCD proteins and/or NA molecules encoding such UCD proteins, with retained therapeutic activity, i.e. enzymatic activity or any other activity that said therapeutic urea cycle proteins carry out. Without wishing to be bound by any theory, it is surmised that these properties are a result of the high content of heat shock proteins, particularly heat shock 70 kda protein 8 (also known as Hsp70-8, encoded for by the gene HSPA8), found in EVs, in particular in exosomes. Other heat shock proteins which may advantageously be present and/or engineered into EVs include Hsp90, Hsp70 and/or Hsp60.

In further embodiments, the EVs as per the present invention are selected to be positive for various protein markers which surprisingly seems to be associated with regenerative and immune-modulatory effects as well as with suitable pharmacokinetics profiles for the treatment of UCDs. The most bioactive EVs are positive for one (but often at least three) of the following polypeptides: CD63, CD81, CD44, SSEA4, CD133, CD24, and various proteins from the heat shock protein family, such as proteins from the Hsp70 family.

Importantly, the therapeutic urea cycle proteins and/or the fusion proteins aiding the loading of NA cargo molecules into the EVs of the present invention are correctly folded, as a result of the endogenous loading of said proteins into EVs. The correct folding is, without wishing to be bound by any theory, surmised to be a result of the heat shock proteins comprised in the EVs, which may help maintain correct folding of the proteins in question.

In yet further aspects, the present invention relates to cells comprising one or more of the polypeptide constructs, the polynucleotide constructs, or the vectors as described herein. Any type of EV-producing cells may be useful for the purposes of the present invention and such EV-producing cells may be present either in vitro, e.g. in cell culture, or in any ex vivo or in vivo system. The cells as per the present invention may optionally be immortalized and/or optionally stably transfected or transduced with at least one polynucleotide construct (or any vector comprising such at least one polynucleotide construct), to enable sustained, robust and consistent production of the EVs.

As abovementioned, in preferred embodiments the EV-producing cells of the present invention are stably transfected and/or transduced with at least one polynucleotide construct(s) which encode(s) for (i) the fusion polypeptide comprising the NA-binding domain and (ii) the NA cargo molecule, or a polynucleotide encoding at least one polypeptide construct comprising a therapeutic UCD protein. In a highly preferred embodiment, the EV-producing cells are exposed to a clonal selection protocol allowing for clonal selection of a single cell clone. Thus, in highly preferred embodiments, the present invention relates to single cell clonal populations of EV-producing cells which are stably transfected and/or transduced to produce EVs comprising both the fusion polypeptide and the NA cargo molecule. The single clones may be obtained using limiting dilution methods, single-cell sorting, single cell printing, and/or isolation of individual cells using cloning cylinders.

In preferred embodiments of the present invention the EV-producing cell comprises at least one polypeptide construct comprising at least one therapeutic urea cycle protein, at least one polynucleotide construct encoding said polypeptide construct and/or at least one vector. The cells of the present invention are typically engineered to comprise the polynucleotide construct (which may be present in the form of a vector such as a plasmid, an mRNA, a linear DNA molecule, a virus or a viral genome, etc.), which is expressed by the cellular machinery into the corresponding polypeptide construct and thereby incorporated into the EVs, normally the exosomes and/or the microvesicles, produced by the cells. Thus, the cells normally initially comprise the polynucleotide construct (or a vector comprising said construct) and once the expression and translation of the polypeptide construct is completed the cell would comprise both the polynucleotide and the corresponding polypeptide constructs, which is normally secreted out from the cell via EV-mediated exocytosis, wherein each and very EV comprises a plurality of copies of the polypeptide construct.

The EV-producing cells of the present invention may preferably comprise a polynucleotide construct, encoding for a polypeptide construct comprising at least one urea cycle protein and at least one EV enrichment polypeptide, which is stably inserted into the EV-producing cell. The creation of a stably (genetically) engineered EV-producing cell source is key to consistent and high-yield production of EVs with a reproducible therapeutic effect and with a reproducible identity from a chemistry, manufacturing and control (CMC) standpoint. The stable cells are normally immortalized, using for instance hTERT immortalization, viral immortalization, and/or conditional immortalization strategies. In order to enable EV production at scale, it is preferable that the EV-producing cells stably comprise the polynucleotide construct (preferably in a suitable vector) over a certain number of population doublings (PDLs), preferably at least 20 PDLs, more preferably at least 50 PDLs, even more preferably at least 70 PDLs, yet even more preferably at least 100 or even at least 200 PDLs.

In a preferable aspect of the present invention at least 50% or 60%, preferably at least 70% or 80%, even more preferably 90% or 95% or more of the EVs produced by the EV-producing cells comprise a polypeptide construct comprising at least one urea cycle protein and/or a polynucleotide construct (such as an mRNA) encoding for at least one UCD protein

In another preferable aspect of the present invention the EVs produced by the EV-producing cells comprise at least 10, 20, 30 or 40 copies, preferably at least 50 copies of the urea cycle protein and/or a polynucleotide construct (such as an mRNA) encoding for at least the UCD protein, more preferably at least 70, 80 or 100 copies.

Thus, in one advantageous embodiment, the present invention relates to compositions comprising a population of EVs, wherein at least 50%, 60%, or 70% of the EVs are positive for a therapeutic urea cycle protein and/or a polynucleotide construct (such as an mRNA) encoding for at least one UCD protein, more preferably wherein at least 75% of the EVs are positive for a therapeutic urea cycle protein or the polynucleotide encoding therefore, even more preferably wherein at least 90% of the EVs are positive for a therapeutic urea cycle protein or the polynucleotide encoding therefore, and/or yet even more preferably wherein at least 95% of the EVs are positive for a therapeutic urea cycle protein or the polynucleotide encoding therefore.

Importantly, the engineering strategies of the present invention for optimizing the EV-producing cells result in a highly efficient loading of the urea cycle protein(s) into the EVs. Typically, each and every EV as per the present invention comprises at least five to ten copies of the polypeptide constructs (and therefore of the urea cycle protein), but more often well above ten copies, for instance around 20-30 copies, or 30-50 copies, or also above 50 copies, for instance around 75 or around 100 copies of the urea cycle protein in question. Clearly, this is highly important for the therapeutic effect and would not be achievable without the purposely selection of optimal engineering strategies and EV profiles, as well as inventive methods for producing and harvesting such EVs. Similarly, the EVs may comprise a polynucleotide construct (such as an mRNA) encoding for at least one UCD protein, preferably in more than one copy per EV, but naturally even more preferably in more than ten copies per EV, or preferably even more copies (such more than 20, 50, or 100 copies per EV). In some embodiments, not all EVs comprises a drug molecule such as an mRNA or a corresponding protein, for instance 1 in 2 EVs may comprise the drug molecule, or 1 in 10 EVs may comprise the drug molecule. Thanks to the safety and tolerability and thereby the wide therapeutic index of EVs, although some of the EVs may not comprise drug cargo the doses of EVs needed to mediate pharmacological effect can easily be achieved by merely increasing the particle (EV) number.

In a further aspect, the present invention further relates to a pharmaceutical composition comprising a plurality of EVs as described herein, at least one polypeptide construct, at least one polynucleotide, and/or at least one vector, and a pharmaceutically acceptable carrier. Importantly, all of the biological components herein (EVs, polypeptides, polynucleotide, vectors, cells, etc.) may advantageously be included in a pharmaceutical composition, either alone or together in any combination. Normally, the pharmaceutical compositions of the present invention comprise a population of EVs and a suitable pharmaceutical carrier, additive, and/or excipient.

In another embodiment, the pharmaceutical compositions may advantageously further comprise pharmaceutical agents such sodium phenylbutyrate or buphenyl, sodium benzoate, lactulose, L-citrulline and L-arginine and/or any derivatives thereof. These types of combinations may result in highly synergistic therapeutic effect, as the EVs deliver a functional urea cycle protein which effect is then potentiated by such pharmaceutical agents. Naturally, the pharmaceutical compositions of the present invention are particularly suited for treating urea cycle storage disorders but other diseases with urea cycle involvement may also be treated using the inventions herein.

The present invention also relates to a method of producing EVs according the invention, comprising: (i) introducing into an EV-producing cell at least one polynucleotide construct according to the invention and (ii) expressing in the EV-producing cell at least one polypeptide construct encoded for by the at least one polynucleotide construct, thereby generating said EVs comprising at least one urea cycle protein, either through direct expression as a UCD protein or via the expression from a polynucleotide that is loaded with the aid of the polypeptide construct. This method is referred to as endogenous loading as compared to exogenous loading. The benefit of endogenous loading compared to exogenous loading of EVs is that it avoids multiple manufacturing steps which result in reduced yield and unnecessary complexity in the drug production process. This improved efficiency of loading applies to both loading of protein and polynucleotide cargos alike. For instance, endogenous loading of native mRNA is much simpler than loading of artificial mRNAs, which typically comprises modified nucleosides, by exogenous loading methods. Furthermore, endogenous loading enables the protein cargos to be properly post-translationally modified before they are loaded into the exosomes. Post-translational modifications are required for proteins to adopt their optimal tertiary or quaternary structure, therefore proteins that are loaded endogenously will be in their optimal confirmation when delivered and therefore have greater therapeutic effect when delivered.

In certain embodiments, a single polynucleotide construct is used whereas in other embodiments more than one polynucleotide construct is employed. Without wishing to be bound by any theory, it is surmised that the EV-producing cell into which a polynucleotide construct has been introduced (either transiently or stably, depending on the purpose and use of the EVs) produces EVs (such as exosomes) that comprise the polypeptide construct encoded for by the polynucleotide. The EVs may then optionally be collected, typically from the cell culture media, and optionally further purified before being put to a particular use. In advantageous embodiments, the EVs produced by said methods further comprise an NA cargo molecule, which is loaded into the EVs with the aid of the fusion polypeptide construct. Typically, a single EV comprises several copies of the NA cargo molecule but a single EV may also comprise more than one type of NA drug cargo molecule.

The EVs of the present invention and/or the pharmaceutical composition of the present invention, can be used for treating one or more urea cycle disorders. Additionally, the pharmaceutical composition according to the invention can also be applied, for use in medicine, preferably for use in the treatment of one or more urea cycle disorders.

In an additional aspect, the present invention can be used to increase the amount of a urea cycle protein in liver, brain and or peripheral cells and/or in any other cellular compartment of a mammal, by a method comprising administering to the mammal a composition comprising either one or more of: (i) EVs, (ii) at least one polypeptide construct, (iii) at least one polynucleotide construct, (iv) at least one vector, and/or (iv) at least one EV-producing cell. Furthermore, the present invention also relates to methods of treatment of an UCD in a subject in need thereof, comprising the steps of: (i) providing a pharmaceutical composition comprising a population of EVs as per the present invention and (ii) administering EVs to a patient. Importantly, as abovementioned, the therapeutic intervention may alternatively comprise administering to a patient either the EVs, the polypeptide constructs, the polynucleotide constructs, the cells and/or the vectors comprising such polynucleotide constructs. This can be carried out using various delivery vectors, e.g. lipid nanoparticles or polymeric or peptide-based delivery vectors. The compositions, the EVs, the polynucleotide and/or polypeptide constructs may be administered to the subject via various administration routes, for instance the EVs as per the present invention may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracerebroventricular, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the EVs, the polypeptide UCD protein and/or the NA cargo molecule in question, or the EV population as such.

The present invention may also be utilized in an in vitro method for intracellular delivery of at least one cargo protein or NA molecule, comprising contacting a target cell with at least one EV according to the present invention and/or at least one polynucleotide construct according to the present invention. Such methods may advantageously be carried out in vitro and/or ex vivo. The methods may comprise the steps of contacting a target cell with at least one EV as per the present invention, or more commonly a population of EVs as per the present invention. Furthermore, the methods for delivery of NA cargo molecules as per the present invention may also comprise introducing into a cell present in any biological system (such as a human being) a polynucleotide encoding for the fusion polypeptides herein.

### EXAMPLE 1

### Loading mRNA into EVs

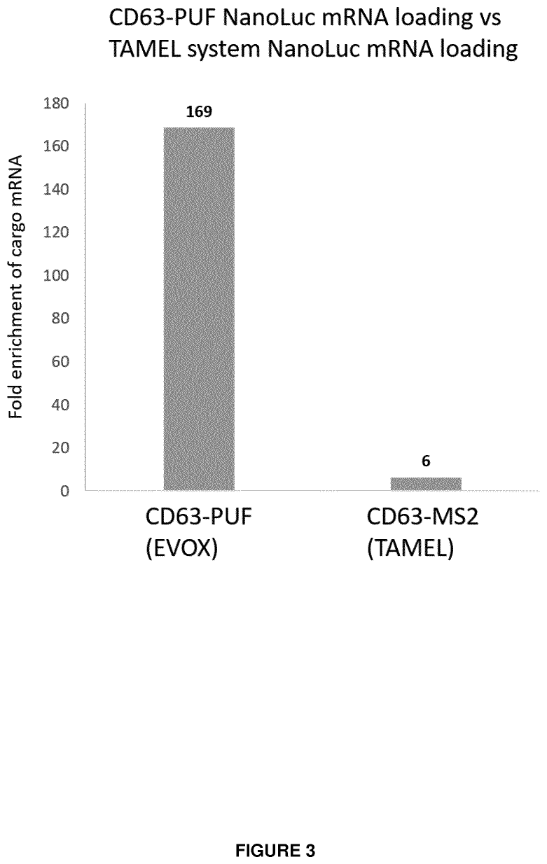

FIG. 3 shows the comparative efficacy of loading a reporter nucleic acid (NanoLuc mRNA) into EVs by an exemplary construct of the present invention (CD63-PUF) compared to the TAMEL loading construct (CD63-MS2).

Cells stably producing a reporter mRNA (NanoLuc reporter mRNA with either a PUF binding site or MS2 binding site incorporated into the mRNA) were further transfected with either CD63-PUF or CD63-MS2 constructs respectively. EVs were produced and purified from these cells and levels of reporter mRNA loaded into the EVs was measured using the NanoLuc reporter. The exosomal protein CD63 is trafficked into the exosomes/EVs and because the exosomal protein is fused to the RNA binding protein (either PUF or MS2 in this case) this results in mRNA with the corresponding binding site being bound by the RNA binding protein and hence being concomitantly loaded into the EVs with the fusion protein.

The CD63-MS2 construct leads to a 6-fold increase in loading of bioactive mRNA into EVs. By contrast the CD63-PUF construct leads to a 169-fold increase in loading of bioactive mRNA into the EVs. Fold increases are calculated compared to loading of housekeeping mRNA, GAPDH. This shows that the CD63-PUF construct achieves significantly improved loading of bioactive mRNA into EVs as compared to the TAMEL CD63-MS2 loading system. As discussed above it is believed that the C63-MS2 loading construct fails to load bioactive mRNA because the mRNA is not released from the tight binding of the MS2 meaning it cannot be translated properly if at all. The data in FIG. 3 shows that the CD63-PUF construct of the present invention over comes this problem and delivers significantly higher levels of bioactive mRNA into EVs compared to the prior art.

### EXAMPLE 2

### Loading Urea Cycle Protein into EVs

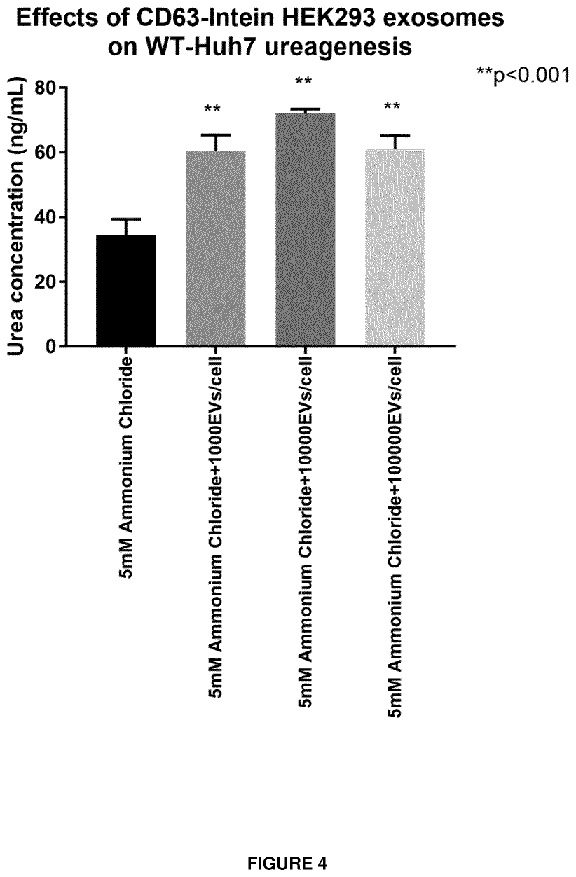

The effect of EVs obtained from HEK cells loaded with the urea cycle protein ALS (Argininosuccinate lyase) on a model of urea cycle disorder was measured using a ureagenesis assay the results of which are shown in FIG. 4.

Ureagenesis Assay Method:

WT-Huh7 cells were cultured in serum free system at 10 k cells per well. EVs from HEK293 cells transfected with CD63-Intein-ASL at 1000, 10000 and 100000 EVs/cell concentration were incubated with the WT-Huh7 cells for 48 h. Samples were washed and incubated with 0.5, 1 or 5 mM ammonium chloride for 24 h. Urea was measured from the supernatant and lysate (data shown from supernatant).

Incubating the cells in ammonium chloride mimics the excess ammonia built up in cells which are deficient in urea cycle enzymes such as those from patients with urea cycle disorders, this is therefore a simple model in which to test protein replacement therapies as disclosed in the present invention.

FIG. 4 shows that cells treated with EVs loaded with ASL produce significantly more urea than un-treated cells. From this it can be demonstrated that the EV treatment supplies bioactive ASL at biologically meaningful levels to the cells which are then able to convert significant amounts of the ammonia into urea using the additional ASL delivered by the EVs. From this it can be seen that EVs loaded with urea cycle proteins have very good potential to enable the treatment of patients with urea cycle disorders by delivering functional urea cycle proteins to cells in need thereof.

### EXAMPLE 3

### Cell-Free ASL Enzyme Activity Assay In Vitro

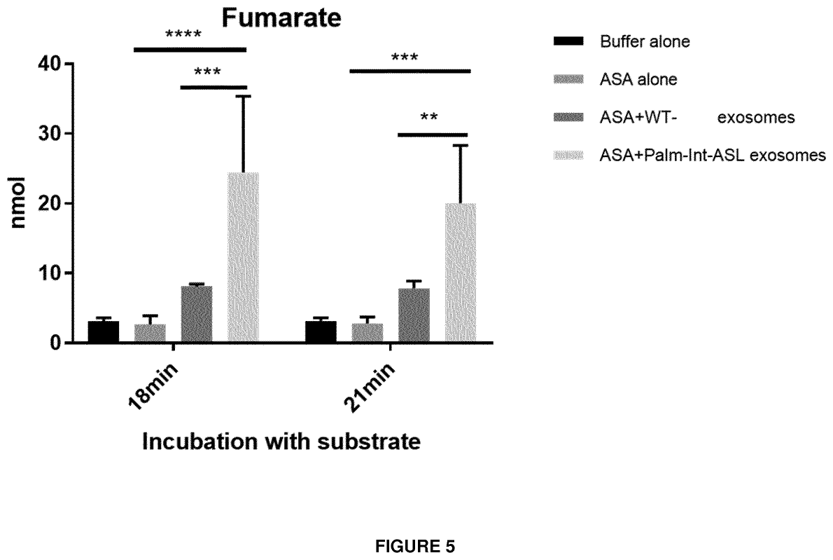

ASL catalyses the reaction of arginosuccinic acid (ASA) to arginine, creating fumaric acid as a by-product. FIG. 5 shows the results of an in vitro, cell free ASL enzyme activity assay. The production of fumaric acid by ASL engineered exosomes vs WT exosomes and ASA control treatment was compared.

The substrate ASA salt (Sigma) was added to preparations which were treated with permeabilising agent (Tween 20) and incubated for 18 or 21 minutes. Fumaric acid levels were detected using a colorimetric kit available from Abcam. As can be seen from FIG. 5 after both the 18 and 21 minute incubations there was a significant increase in the amount of fumarate produced by the exosomes engineered to contain ASL-Palm-intein when compared to ASA alone or WT exosomes. This shows that the ASL protein was active and released by the cleavage of the intein.

### EXAMPLE 4

### In-Vivo ASL Activity Assay

ASL knock-out mice were dosed at day 15+/−1 day with exosomes loaded with ASL (as part of a Palm-Intein-ASL or CD63-Intein-ASL construct), WT exosomes or vehicle treatment. Blood ammonia levels were then tested using an ammonia assay kit (Sigma). The ASL knock-out mouse model exhibits increased blood ammonia levels which is a symptom shared by many urea cycle disorders.

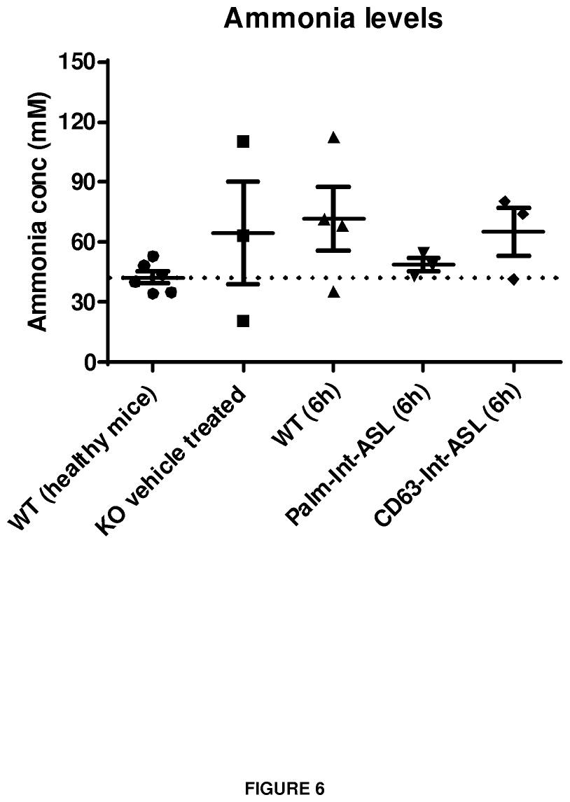

FIG. 6 shows clearly that exosomes engineered to contain the Palm-intein-ASL construct or CD63-intein-ASL construct were capable of lowering blood ammonia levels. Particularly the palm-intein construct was capable of lowering the blood ammonia levels to levels similar to those of WT mice. This shows that the ASL protein when delivered by exosomes in-vivo was biologically active and in-vivo delivery of exosomes loaded with ASL was capable of restoring the blood ammonia levels of KO mice to healthy levels after only a single treatment.

1. An extracellular vesicle (EV) for replacement of urea cycle proteins, characterized in that the EV is engineered to comprise at least one urea cycle protein and/or at least one polynucleotide encoding for a urea cycle protein.

2. The EV according to claim 1, wherein the urea cycle protein or the polynucleotide encoding for a urea cycle protein encodes for a protein selected from the group comprising: argininosuccinate lyase (ASL), arginase, mitochondrial ornithine transporter, argininosuccinic acid synthase, N-acetylglutamate synthase, carbamoyl phosphate synthetase, ornithine transcarbamoylase, citrin, y+L amino acid transporter 1, uridine monophosphate synthase or any fragments thereof, derivatives, domains, or combinations thereof.

3. The EV according to claim 1, wherein the urea cycle protein is comprised in a fusion polypeptide comprising an EV enrichment polypeptide.

4. The EV according to claim 1 comprising a fusion polypeptide comprising an EV enrichment polypeptide and a nucleic acid (NA)-binding domain.

5. The EV according to claim 4 wherein a polynucleotide encoding for a urea cycle protein is transported into the EV with the assistance of the NA-binding domain of the fusion polypeptide.

6. The EV according to claim 5 wherein the polynucleotide may be an mRNA, a viral genome or a plasmid encoding at least one urea cycle protein.

7. The EV according to claim 4, wherein the NA-binding domain is one or more of a protein from the PUF family, a protein from the Cas6 family, a protein from the Cas13 family, and/or a nucleic acid aptamer-binding domain or any fragments thereof, derivatives, domains, or combinations thereof.

8. The EV according to claim 3, wherein the EV enrichment polypeptide is selected from the group comprising CD9, CD53, CD63, CD81, CD54, CD50, FLOT1, FLOT2, CD49d, CD71, CD133, CD138, CD235a, ALIX, AARDC1, palmitoylation signal (Palm), Syntenin-1, Syntenin-2, Lamp2b, TSPAN8, syndecan-1, syndecan-2, syndecan-3, syndecan-4, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, other exosomal polypeptides, and any fragments, derivatives, domains or combinations thereof.

9. The EV according to claim 3, wherein the fusion polypeptide further comprises an intein.

10. The EV according to claim 1, wherein the EV further comprises at least one heat shock protein.

11. The EV according to claim 1, wherein the EV comprises at least one urea cycle protein which is substantially correctly folded.

12. The EV according to claim 1, wherein the EV further comprises at least one tissue targeting moiety capable of targeting the EV to a tissue or organ of interest.

13. A polypeptide construct comprising an EV enrichment protein fused to a urea cycle protein.

14. The polypeptide construct of claim 13, wherein the fusion protein further comprises an intein.

15. A pharmaceutical composition comprising:
(i) at least one polypeptide construct according to claim 13, and/or
(ii) at least one EV according to claim 1,
and a pharmaceutically acceptable excipient or carrier.
